(19) United States
(12) Patent Application Publication
(10) Pub. No.: US 2023/0253104 A1
(43) Pub. Date: Aug. 10, 2023

(54) SYSTEMS AND METHODS FOR MOTOR FUNCTION FACILITATION

(71) Applicant: Thomas Jefferson University, Philadelphia, PA (US)

(72) Inventors: Mijail Demian Serruya, Merion Station, PA (US); Alessandro Napoli, Philadelphia, PA (US)

(21) Appl. No.: 18/014,613

(22) PCT Filed: Jul. 9, 2021
(86) PCT No.: PCT/US21/41087

§ 371 Date: Jan. 5, 2023

Related U.S. Application Data
(60) Provisional application No. 63/049,754, filed on Jul. 9, 2020.

(51) Int. Cl. G16H 40/63; G06N 5/022; A61N 1/36; A61F 2/72

(52) U.S. Cl. CPC G16H 40/63; G06N 5/022; A61N 1/36003; A61N 1/36031; A61F 2/72

(57) ABSTRACT

Systems and methods for motor function facilitation are described herein. In one aspect, a computer-implemented method for assisted actuation of a patient movement can include: receiving a set of neural signals from a set of neural sensors; extracting a set of features from the set of neural signals; inputting the set of features into a classification model; determining from the classification model an attempted activity of a user; and transmitting a set of stimulation signals to one or more output effectors according to the attempted activity and the set of neural signals.

### CROSS-REFERENCE TO RELATED APPLICATION(S)

This application claims priority to U.S. Provisional Patent Application No. 63/049,754, filed Jul. 9, 2020, which is incorporated in its entirety herein by reference.

### BACKGROUND OF THE INVENTION

Conventional physical therapy techniques require extensive face-to-face interaction between a patient and therapists. For example, the therapist instructs the patient on how to perform a certain movement, the patient attempts to perform the movement, and the therapist responds with an assessment (e.g., how to perform the movement better). This interaction is repeated, and as the patient's physical condition changes, the therapist's instructions are altered to adapt to the patient's condition.

While automated assistive rehabilitation systems and techniques are in their infancy, these systems and techniques suffer similar issues. For example, while a rehabilitation system can be utilized offsite by a patient, the patient still must return onsite periodically to provide data to the therapist or system manager, who then can calibrate the system according to the needs of the patient.

Further, conventional rehabilitation systems and techniques fail to identify some modes of movement for a patient. Some patients may lose the ability to traditionally actuate a movement. However, the patient may still retain secondary modes of movement that may be initiated by a patient during an attempted movement, but the secondary mode is insufficient on its own to actually perform the movement.

### SUMMARY

Systems and methods for motor function facilitation are described herein. In one aspect, a computer-implemented method for assisted actuation of a patient movement can include: receiving a set of neural signals from a set of neural sensors; extracting a set of features from the set of neural signals; inputting the set of features into a classification model; determining from the classification model an attempted activity of a user; and transmitting a set of stimulation signals to one or more output effectors according to the attempted activity and the set of neural signals.

This aspect can include a variety of embodiments. In one embodiment, the computer-implemented method can further include training the classification model, where the training includes: receiving a set of training neural signals from the set of neural sensors; receiving input indicative of an action performed by a trainer; extracting a set of training features from the set of training neural signals; and mapping the set of training features to the indicative action.

In some cases, training the classification model further includes determining a feature value threshold from the mapping, where the attempted activity of the user is further determined from a feature of the set of features reaching the feature value threshold. In some cases, the trainer can include the user, a provider of physical therapy, a provider of occupational therapy, or a combination thereof.

In another embodiment, the computer-implemented method can further include identifying a set of proportional values between the set of neural signals and the attempted activity of the user; and generating the set of stimulation signals according to the set of proportional values.

In some cases, identifying the set of proportional values is effected via a multilayer perceptron network, a convolution neural network, a genetic algorithm, a binary particle swarm optimization process, a generative adversarial network, a support vector machine of polynomial and radial basis kernel, a Kalman filter, a generalized linear mixed model, a particle filters, a random forest algorithm, a rotation forest algorithm, or a combination thereof.

In another embodiment, the computer-implemented method can further include identifying an activation pattern from the received neural signals, wherein determining the attempted activity is according to the identified activation pattern.

In another embodiment, the computer-implemented method can further include modifying the classification model according to the set of features.

In another embodiment, the set of neural signals can include scalp EEG, subgaleal EEG, intraosseous EEG, epidural EEG, subdural EEG, intracortical LFPs, depth EEG, single unit recordings, heart rate, heart rate variability, respiratory rate, galvanic skin conductance, blood sugar level, pupil diameter, extraoculogram, electromyogram, positioning of a user body part, user's kinematic and kinetic signals, sound signals, keyboard entry, mouse click, joystick use, or a combination thereof.

In another embodiment, the output effector can include a set of electrical contacts, an electrical prosthetic, a brain-computer interface, or a combination thereof.

In another aspect, a system can include a neural signal processor configured to: receive a set of brain signals from a user; digitize the set of brain signals; and store the digitized brain signals in a buffer; a neural signal analyzer configured to: retrieve the digitized brain signals from the buffer; identify a set of spike counts, local field potentials (LFPs), or a combination thereof, from the digitized brain signals; extract a set of features from the set of spike counts and LFPs; input the set of features into a classification model; identify from the classification model an attempted motor movement of the user; generate a motor control command according to the attempted motor movement; and transmit the motor control command; and a rehabilitation prosthetic configured to: receive the motor control command; and generate a corresponding motor movement according to the motor control command.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a system for motor function facilitation according to an embodiment of the present disclosure.

FIGS. 2-4 illustrate workflow processes for motor function facilitation according to embodiments of the present disclosure.

FIGS. 5 and 6 depict graphical user interfaces for the motor function facilitation system according to embodiments of the present disclosure.

FIG. 7 depicts Fast Fourier Transform (FFT) results from received electronic input signals of the motor function facilitation system according to an embodiment of the present disclosure.

FIGS. 8 and 9 depict graphical user interfaces for a virtual arm reality arm operator according to embodiments of the present disclosure.

FIG. 10 illustrates a high-level overview of a motor function facilitation system according to an embodiment of the present disclosure.

FIGS. 11 and 12 illustrate system architectures for motor function facilitation systems according to embodiments of the present disclosure.

FIGS. 13-15 illustrate software architecture for motor function facilitation systems according to embodiments of the present disclosure.

FIG. 16 illustrate software architecture for a wireless controller module according to an embodiment of the present disclosure.

FIGS. 17-23 illustrate software architecture for motor function facilitation systems according to embodiments of the present disclosure.

FIG. 24 illustrates a physical layer architecture for a motor function facilitation system according to an embodiment of the present disclosure.

FIGS. 25 and 26 illustrate motor function facilitation systems according to embodiments of the present disclosure.

FIG. 27 depicts a clinical trial timeline implementing motor function facilitation systems according to embodiments of the present disclosure.

FIG. 28 depicts a motor function facilitation system according to an embodiment of the present disclosure.

FIG. 29 depicts neuroimagaing results for the patient participating in the clinical trial. The results depict diffusion sequence when the acute stroke occurred; diffusion restriction is evident in the right lentiform nucleus and adjacent white matter (Panel (a)). T2-weighted Mill two years later shows areas of encephalomalacia and relative ventriculomegaly (Panel (b)). Functional neuroimaging revealed a hot spot of activation, indicated by a circle, in the depth of the central sulcus along the ‘hand knob’ area of the precentral gyms (Panel (c)). A three-dimensional reconstruction of the participant's cortical surface derived from Mill with imagined left hand movement centroid of activity indicated by the circle (Panel (d)). Shading indicates an area responsive to sensory stimulation of the left hand. Squares indicate microelectrode arrays.

FIG. 30 depicts action potential waveforms recorded from the participant patient according to an embodiment of the present disclosure.

FIG. 31 depicts neuronal activity correlated with performed movements in the paretic limb. Over a 110-s period, the participant was asked to perform a series of left limb movements (described on abscissa). Verbal movement instructions indicated by hash marks. Rasters indicate the time of each action potential. Normalized, integrated firing rates appear beneath each raster, derived by a ‘leaky integrator’ equation; normalization achieved by dividing by the maximum integrated firing rate from each unit's spike train over the time period displayed. The top unit (channel 61) is more active for hand squeezing than wrist extension, relative to the bottom, simultaneously recorded unit (channel 62). The participant performed all movements: such motions required effort and he was unable to engage a consistent level of activity for each cue and exhibited a variable reaction time. The participant was easily fatigued, requiring him to take a break and adjust posture.

FIG. 32 depicts cumulative, integrated spike activity across channels fluctuating with joint position (top graph) and residual left forearm electromyographic activity (bottom graph). The summed spike activity across channels and run through a leaky integrator appeared to fluctuate with specific residual actions in the left upper extremity. Proximal residual activity generated a normal-appearing pattern: as seen between 290 and 310 seconds in the bottom panel, biceps and triceps activity alternate. In the distal upper extremity, however, wrist flexor and wrist extensor activity tend to occur together in an abnormal synergy; also, wrist flexor activity is abnormally synergistic with biceps activity (an abnormal flexor synergy). The summed, integrated spiking activity across channels appears to covary with wrist flexor activity.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Motor Function Facilitation System

The motor function facilitation system can include a motor functional apparatus with modular components, software, and a calibration protocol. The motor function facilitation system can be used to restore independent voluntary movement in the hands, arms, trunk and legs in adults and children with weakness or paralysis due to neurological disease or injury.

The software infrastructure and calibration approach of the motor function facilitation system can be extended to other types of treatment, including rehabilitation and daily functional assistance for people with cognitive disorders due to neurological disease and injury; as an adjunct to mental illness treatment by incorporating principles of cognitive behavioral, dialectic emotional, mindfulness-based, and hypno-therapies; and treatment for non-neurologic conditions that merit long-term or telehealth mediated follow-up such as for orthopedic or cardiac post-operative rehabilitation.

The motor function facilitation system can be deployed as a medical device designed to treat adults and children with neurological disease and injury such as paralysis or paresis or incoordination due to spinal cord injury, stroke, ALS, TBI, MS, muscular dystrophy, neuropathy, transverse myelitis, brachial plexus injury, amputation, tumor resection, or such as cognitive impairment due to autism spectrum, Alzheimer's disease, TBI, stroke, Parkinson's, DLB, FTD, MSA, PSP, CBD, PPA, MS, CP, chromosomal disorders, and the like. Further, the motor function facilitation system can also be used as part of rehabilitation, functional restoration or both, for children and adults with weakness, inattention, fatigability or other symptoms due to non-neurological conditions, such as chemotherapy for cancer or autoimmune conditions, recovery from cardiovascular conditions or surgeries, and orthopedic interventions.

The motor function facilitation system can be deployed as a consumer product for healthy people to provide a novel input device, to enhance performance in military or industrial settings, for athletic skill refinement, or for entertainment. In addition, the system's software and calibration protocol can be used to provide control of phone, computer and other devices, as well as for text entry, communication and productivity for able-bodied children and adults, (e.g., via mechanical, electromagnetic and other wearable sensors).

The motor function facilitation system can include an optimal network of devices and tools to:

1) meet the user's needs;

2) achieve the user's functional objectives;

3) incorporate the user's preference in device use/integration and technology (e.g., such as whether the person wants to wear components all the time or only wear it in specific situations);

4) incorporate incremental adjustments in complexity that are informed by the outcomes achieved by use over time.

A key innovation of the motor function facilitation system is that it can function as a service, rather than a static device. In this sense the motor function facilitation system can be a “living service” with interchangeable modular components continually responding to the user's needs and incorporating changes in the user's abilities and goals. The motor function facilitation system can be a scalable and flexible platform that allows for the integration of multiple neuromodulation devices, sensors and software tools.

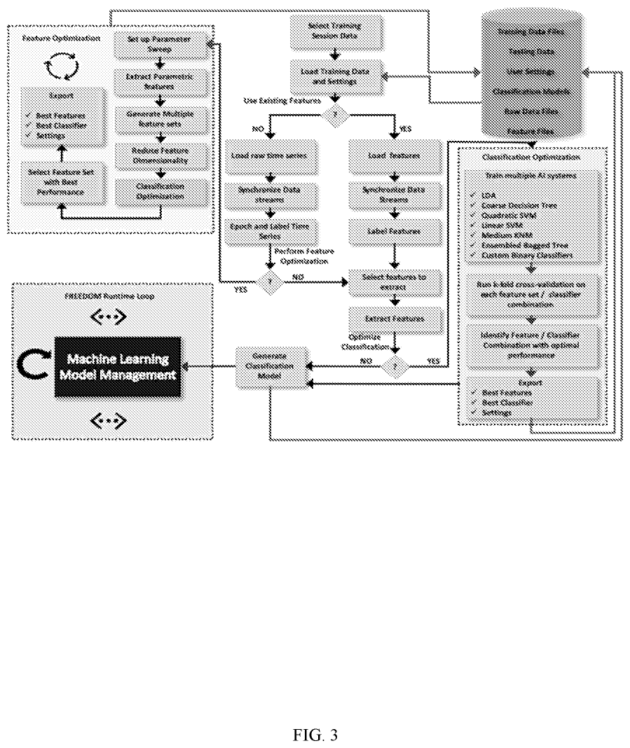

The software of the motor function facilitation system can manage, control and operate a network of commercially available devices and sensors, with the goal of achieving optimal rehabilitation/assistive results. Further, the motor function facilitation system can be a modular, flexible, scalable and expandable closed-loop system, capable of sensing patient-specific physiological signals and using these to generate the optimal control and activation commands to a network of patient-specific neuromodulation tools. FIG. 3 depicts a workflow for feature optimization and classification optimization processes implemented by the motor function facilitation system. The motor function facilitation system can be highly customizable based on medical and patient's needs. The motor function facilitation system can be used in multiple settings, including at home with tele-health support. To facilitate home use, the motor function facilitation system software can feature a patient-specific User Interface (UI). This UI can provide for the necessary daily and task-specific adjustments (parameter setting and stimulation type and dose) without the need of changing the individual neuromodulation devices' settings.

### Components

The motor function facilitation system can include a number of assistive actuators, electric stimulators, and/or electrical signal sensors. For example, the motor function facilitation system can include or integrate the following examples of wearable powered devices into the system: OmniHi5™, Myomo®, and MyoPro™ motorized orthoses; upper extremity rehabilitation/feedback systems such as MyndMove™; lower extremity functional systems such as WalkeAide®; whole-body exoskeletons such as SuiteX™ and Ekso®; lower extremity rehabilitation and functional mobility systems such as Myolyn® and MyoCycle™; Empi™ portable neuromuscular electrical stimulator; Cyclone Xcite portable multi-channel FES therapy system; Zynex Medical Neuromove; Bioness, L300, L300 Plus and H200 systems; Saebo MyoTrac Infiniti biofeedback electrical stimulator, and the like.

### Software Environment and Graphical User Interface

The motor function facilitation system can inter-connect with user performance tracking systems, and can link to software application programs (apps) such as calendars, alarms, email, reminders, medication management, and the like. Software of the motor function facilitation system can provide entry of information about a particular user such as medical history, learning history, and prior test results such as for motor tests, neuropsychological tests or other tests. The software can access normative databases on particular tasks and previous baselines of the user to generate standard deviations and other scorings of performance on a use-by-use or calibration-by-calibration basis. The software can display visual information (e.g., images, videos, and the like) either on a display screen, such as on a left-hand side or the whole screen. In some cases, corresponding text can be displayed as well (e.g., on the right side of the screen) and audio (e.g., voice instructions) can be played on the speaker of the computer or mobile device.

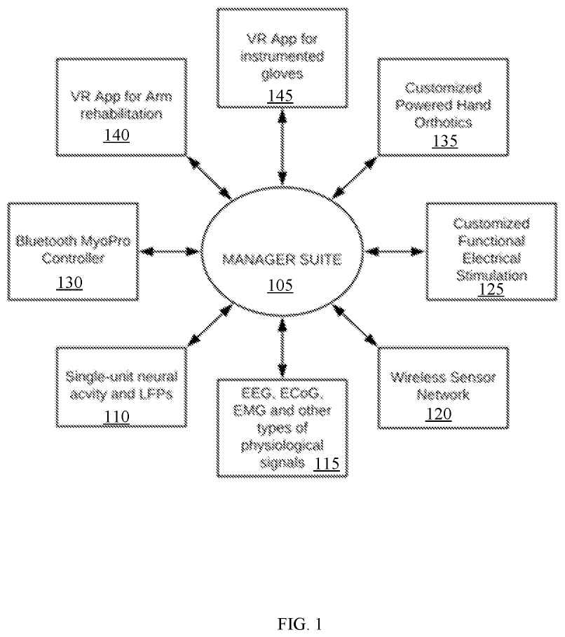

FIG. 1 illustrates a motor function facilitation system according to an embodiment of the claimed invention. The system can include a manager suite 105. The manager suite 1-5 can receive sensed electrical data, inputted setting parameters and other operational features, such as operational features for a Bluetooth wireless controller 130 with input/output components. In some cases, the wireless controller can implement over wireless telecommunication protocol, such as Zigbee, and the like.

The system can also include various electrical data sensors (e.g., which transmits neural activity 110, physiological signals 115, electrical stimulation 125, hand orthotics 135, instrumented gloves 145, arm rehabilitation unit 140, and the like). In some cases, the sensors can include a wearable (e.g., chronical or intermittent wearable) sensor for the user, along with a microcontroller and signal acquisition. Batteries can power the sensor, controllers, wireless components and motors.

Some units in the system can also act as output effectors. For example, arm rehabilitation unit 140, instrumented gloves 145, hand orthotics 135, and the like, can also transmit electrical signals to a user (e.g., via the manager suite 105)

### Input Sources

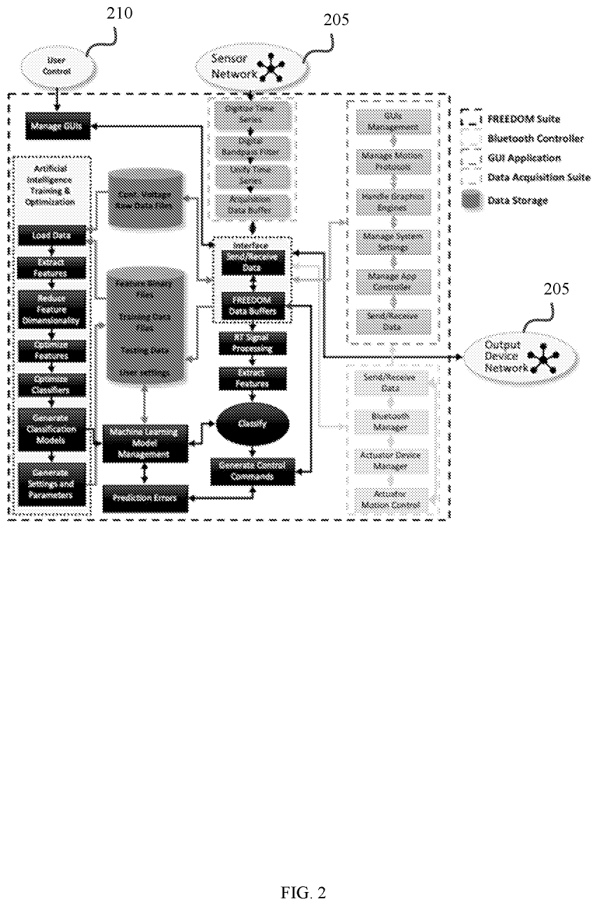

FIG. 2 depicts an exemplary motor function facilitation system (e.g., the FREEDOM system) according to embodiments of the claimed invention. The system can include wearable or implanted sensors (e.g., sensor network 205). Wearable or implanted sensors can include electrical neural sensors (e.g., low impedance EEG, high impedance microelectrodes as individual wires or in arrays, living constructs; with sensors at the scalp, subgaleal, intraosseous, epidural, subdural, intracortical or depth locations, and the like), electrical cardiovascular sensors (ECG for heart rate or heart rate variability), neuromuscular sensors (EMG), mechanical switches, respiratory rate sensors, galvanic skin response sensors, temperature sensors, accelerometers/gyroscope (external wearable or implanted), camera, microphone, keyboard, mouse, touch pad, touch screen, joystick, off the shelf user input device, motion capture sensors, quotient limb, head or eye jitter, eye movement (visual or infrared camera or EOG) sensor, pupil diameter, decoded facial state sensor, voice analysis sensor, and the like. User controls (e.g., user control 210) can include, mouse, actual keyboard/keypad, virtual keyboard/keypad, hand gestures, actual/virtual button clicks/dwells, cursor trajectories, cursor locations, sip/puff, EMG controllers, voice activation, and the like. The physiological/electrical signals captured by these sensors may be referred herein as “neural signals.”

The motor function facilitation system can be extensible, as input sources can be removed or added dynamically. For example, the user can experience more difficulty using certain surface EMG input signals such that they are removed as a possible input, while a new external or implanted sensor can provide a new input source that can be added.

### Output Effectors

The motor function facilitation system can also include output effectors (e.g., output device network 215). Once signals are recorded and decoded (e.g., by the manager suite 105), the signals can be deployed to cause some action to occur. This deployment can include display text, images, or video displayed on a screen (desktop, laptop, phone), or sound (verbal, music), and even olfactory cues. Vibrotactile feedback output can include wearable, implanted or device tactors (chair, phone) or electrical stimulation (haptic ‘phosphene’).

To restore movement, two of the most frequently used output effectors are motor actuation and electrical stimulation. A motor placed on a rigid brace with mobile rod components can cause hinge joint movement. Electrical stimulation can be used to cause an underlying muscle or group of muscles to contract (functional electrical stimulation). These two approaches can be used independently, to achieve movement across the same joint, or can be used simultaneously in parallel at one or different joints (e.g., elbow and wrist).

When a given output is triggered, it can be binary (e.g., motor goes from one position all the way to another position), continuous (proportional), or a more complex pre-programmed sequence. The motor function facilitation system allows pre-programming of stimulations that target different movements and various muscles. The protocols can involve input by a medical provider (e.g., physical or occupational therapist) in order to ensure correct setup. The system's calibration can overcome limitations imposed by other pre-programmed functional electrical stimulation systems on the market. For example, the motor function facilitation system can alter pre-programmed motions in terms of motion quality control and timing of individual muscle groups. The muscle activation sequences can be adjusted to individual patient's needs without a consistent manual intervention from a therapist that other systems require. The motor function facilitation system can automate outputs for specific users in a customized manner without intensive manual setting and control to be operated. Further, the motor function facilitation system can incorporate the use of multiple sensors, some for control, and some for feedback for the system itself (e.g., strain gauges, potentiometers, push-buttons, capacitive buttons, and accelerometers for joint angle), allowing the system to be closed-loop and to operate without a medical therapist present. Additionally, the motor function facilitation system allows for removal or addition of output effectors as appropriate in a dynamic manner.

One or more orthotic or prosthetic components can be implemented in the system (e.g., for actuation or stimulation). These components can be purchased off-the-shelf or custom-made using 3D printing techniques for the user, including the same user over time (such as children growing into adults).

### Decoders and Mappings

The closed-loop nature of the motor function facilitation system allows for dynamic and efficient stimulation methods and real-time integration of different technologies. Artificial Intelligence (AI) can update and revise parameter and output controls as a patient continues to use the system. The system can continue to search for optimal settings based on patient performance data. Further, an alert system can be implemented to ensure treatment efficacy and patient safety. In rehabilitation settings, the system can provide automatic feedback and task guidance, thereby allowing patients to have more time to effectively train. This can allow medical service providers to spend time with patients focusing on intervention and therapy rather than having to observe repetitions of the same task. The closed-loop nature of the system can also provide medical service providers and insurance payers with quantitative outcome metrics that can be used to: 1) inform intervention protocols and 2) keep track of changes and progress.

The motor function facilitation system can also trigger electromagnetic stimulation using a variety of device components, at a variety of anatomical sites, and using a variety of settings. Components can include metal contacts, disc electrodes, soft conductive pads, living constructs, and the like. Further, stimulation can include transcranial stimulation at the scalp, subgaleal stimulation, subdural stimulation, depth stimulation, spinal stimulation, skin stimulation including at peripheral sites (e.g., median nerve, posterior tibial nerve), vagal nerve stimulation (VNS implanted or external at neck or auricular branch), and the like. Current can be delivered directly, via the stimulation components, as alternating, variable, random noise bias, or frequency sweeps. The system can also implement multi-site stimulation, which can include intersection short-pulses or temporal interference.

In addition to simply triggering stimulation initiation or termination at a given site, the motor function facilitation system can analyze decoded signals to modulate the stimulation parameters such as pulse width, pulse shape, pulse polarity, pulse amplitude, frequency, duration, grouping (single pulse, train, frequency sweep), which contacts serve as source and sink, and the like. In some cases, the motor function facilitation system can also be used to trigger magnetic stimulator control.

The motor function facilitation system can be configured to apply a given type of stimulation (electrical, mechanical, thermal, etc.) based upon the stage of a functional task, for example during the particular phase of a rehabilitation exercise, when a timer set by the user is completed, when receiving a decoded signature of fatigue or engagement, at particular verbal, visual or tactile cues or instructions, or when sensors note a task situation (e.g., the hand is next to a water bottle instrumented with an RFID chip to alert the orthoses to prepare the hand to grasp it, or non-contact sweeping the hand by a wall switch to activate it) and the like.

The motor function facilitation system can also adjust the settings of any given input signal or output source. For instance, input signals recording muscle activity can be averaged into a root mean square. Likewise, the user, the medical service provider, or the software can set a threshold for the input signal to cross to trigger a given output. The output range of a given effector can be arbitrarily constrained or expanded (such as a range of allowable current amplitude for FES, motor angle displacement, or net joint change by accelerometry or strain gauge feedback, etc.). The system software can also provide a user or service provider the ability to apply certain scaling and transform rules onto inputs and outputs (e.g., a given input signal can be band-passed, scaled by a multiple, offset by a coefficient, or convolved with filters of arbitrary shapes such as a sigma transform common in sensory physiology).

System decoders can be configured to be deployed in real-time. Thus, recorded signals can trigger various output actions continually in real-time as the user is engaged in daily life and a variety of activities. While offline analysis can be performed by a technician or medical providers, the motor function facilitation system can also achieve functional movements that are performed in the moment.

### Discrete Decoding

Discrete decoding refers to receiving a given input signal and mapping the signal into one or more discrete output states. For example, an input signal can be mapped on to a particular motion, such as the elbow fully extended or fully flexed. More than one input signal can be combined to decode a discrete state, and a given decoded discrete state can achieve more than one effect (for example, if the state ‘move hand towards head’ is decoded, multiple motor effectors can be activate to achieve the desired motion).

The motor function facilitation system can map any arbitrary assignment between inputs and outputs in a discrete manner. When input signals are combined, or when a given input signal is noisy or complex, a formal decoding algorithm can be deployed. In some cases, a Bayesian decoder may be implemented, which can combine calibration data and a priori probabilities of given actions. Constrained by various inputs; the software of the system can be taught contextual states (e.g., a practice state, at-home-daily state, school-work state, task-specific state, and the like). The motor function facilitation system can also include a graphical display for the user to view decoder output (e.g., before the output is mapped on to an effector).

### Continuous Decoding

In addition to decoding discrete states, the motor function facilitation system can contain an expandable bank of algorithms to identify continuously varying output states. Hence, instead of simply making a trigger rule based on a threshold (e.g., the root mean square of an EMG signal exceeds a predefined threshold), a continuous decoder can vary the output in a proportional scaled range. For example, the amplitude of an EMG root mean square can be scaled into a precise joint angle continuously rather than all-the-way flexed or all-the-way extended). Decoder options can include multilayer perceptron networks, convolution neural networks, genetic algorithms, binary particle swarm optimization, generative adversarial networks, support vector machine of polynomial and radial basis kernel, Kalman filters, generalized linear mixed models, particle filters, random forest, rotation forests, and the like. Principal and independent component analyses can be deployed in both discrete and continuous decoders.

### Rapid State Space Search

In addition to mapping input signals to output effectors, the various discrete and continuous decoders can also search the state space of the input signals to constrain subsequent calibration. In some cases, a calibration procedure can include having a user imagine, attempt and (when physically able) perform specific actions. In other cases, a calibration procedure can include a user observing another individual or virtual avatar performing an action. In some cases, a calibration procedure can include output effectors passively “dragging” a user. However, additional control signals can be derived by having the user imagine, attempt to perform certain postures and sequences. Likewise, certain signals (such as small motor units detected by independent component analysis in high density trapezius surface EMG) in some cases may not have an obvious or conscious control correlate, and by requesting particular imagined/attempted movements, a user's activation patterns may be induced as valuable for decoding in real-time. Generative adversarial networks, random forest and all the other algorithms already cited can be configured to generate an appropriate instruction set of target movements or other timestamped goals and tasks.

### Feedback-Based Error Minimization Re-Calibration

In certain cases, a user can deploy the motor function facilitation apparatus for functional tasks as soon as a preliminary mapping or collection of mappings are made from input sources to output effectors. In other cases, there may be a significant advantage to repeating certain calibration steps after an initial calibration for error minimization. This approach can provide rapid re-calibration of the system such that the user can practice using the effector and the technician or provider may use the data collected during these initial practice attempts to identify a new set of discrete or continuous decoder parameters (such as linear filter coefficients). The motor function facilitation system can in some cases periodically query the user, technician or provider regarding whether to repeat a calibration or a re-calibration session.

### Calibration

Calibration refers to the process of the system registering active input options and output options and adjusting the mappings such that the user can control the various outputs to achieve functional goals. The weighting and transformation of the mappings can occur at a sensor by sensor level or by combining all inputs together in aggregate. The calibration can take into account previously programmed macros or other pre-programmed routines provided by a health provider, family member, the user, or automatically generated by the system itself. Further, the calibration process can occur automatically, initiated by the user (or group of users), a technician, a health provider, a friend or family member or caregiver designated by the user, or a combination of these options.

A graphical user interface can be used in the calibration procedure to receive input. The system can then map inputs to outputs, including meta-modulating outputs such as to the brain, peripheral nerves or spine that can bias net movements.

Data processing performed on any or all of the above can include power spectra Fast Fourier transform, wavelet convolution, Hilbert transform, Bayesian classifiers, filters (linear, particle, Kalman or hybrid), hysteresis tracking, and the like. The motor function facilitation system can track and timestamp the stage of every calibration, training and functional restoration task and any biomarker signature or provider-tracker (presenting new information, encoding-storage-retrieval, learning vs testing, cognitive math-language-per-customized vocation) and the task performance (personal score, performance thus far relative to different baselines, prior sessions, normative data sets).

The calibration procedure can occur with or without a technician present, and with or without a technician available remotely. Screens can display data (to health providers, the user, and designated caregivers) about the user's health status and medical condition. The calibration can involve the systematic identification of desired and available inputs and particular sensors by their signatures. If certain sensors require mechanical adjustment (for example a conductive contact with excess impedance), the system can generate alerts and instructions for the corresponding adjustment. In addition to taking into account the time series and trigger data from the various sensors or pre-programmed routines, and ongoing annotation by the user or technician or others, the system can also track other performance features, for example, 300 millisecond post instruction positive deflection on the EEG (P300), peak alpha frequency, queries to the user on insight, subsequent memory recall, arousal via EEG/EMG/EKG and other metrics, cognitive effort, and the like. Further, the calibration can involve 2 and 3D animations, videos and virtual reality displays, sounds, and tactile feedback and may deploy content salient specific to the particular user, such as music, sounds, personalized, photos, and other images. The signal quality of sensors can be continually tracked in the background both during the calibration and throughout use, such as electrically conductive sensor impedance and mechanical switch positioning.

Calibration can be tailored to specific task and can be “meta” to each task. Hence, there can be regularly scheduled calibration sessions that incorporate diaphragmatic breathing, guided imagery, relaxation exercises, hypnotic suggestions, eye movements, and specific types of biofeedback. This type of meta-calibration both can help optimize the settings and mappings between inputs and outputs, and help train the user to optimally deploy the system in a variety of settings. Biofeedback can include EEG-based neurofeedback to titrate up or down arousal to optimize learning of functional tasks.

Calibration can also provide for an environment for testing outputs for a variety of tasks. Physical elementary motor tasks (e.g., hand opening and closing) can be coupled to functional tasks (grasping a cup) and also cognitive tasks (speaking a word in a new language, such as pronouncing the word for ‘cup’ in a new language). Cognitive versions of the motor function facilitation system can steer calibration towards tutoring of new material in mathematics, language, engineering, finance and other topics.

A rapid quantitative method for mapping visual receptive fields can be adapted for other sensor input. Bayesian active learning methods, including a utility function that selects stimuli to minimize the average posterior variance of sensor input, can analyze the relationship between prior parameterization, stimulus selection, and active learning performance. Another approach can include stochastic gradient descent and generalized linear classification schemes. Rapid serial visual presentation can be deployed in calibration to allow the participant and the software to identify and detect optimal mappings.

The calibration system can guide or “walk” a user through a variety of daily activities (e.g., motor-based, such as lifting a laundry basket, brushing teeth, picking up glass and drinking, etc.; and/or cognitive-based, such as filling out taxes, performing a banking action, medication management, etc.).

Calibration can also use cues for facilitating the completion of tasks. Examples of cues can include vanishing cues, olfactory cues, tactile cues, vibrotactile or electrotactile artificial context, vibrotactile or electrotactile paired associates, spaced intervals, purposefully-altered task settings, multi-modality poly-sensory feedback, teaching metaphors, mnemonics, method of loci, motor learning, teachbacks, purposeful alternation of focused intense practice with relaxation, yoga postures, qi-gong postures, permissive mind-wandering intervals, and the like.

### Fast, Reliable, Effortless, Elementary (FREE) Input Search

The motor function facilitation system includes a method determine a user's optimal free signals. For a person with motor impairment there exists, somewhere in their brain or body, intact signals that can be used to control devices to restore movement. If somebody has a paralyzed hand for example, they may have intact control of the elbow such that elbow motion could be used to trigger a device that restores movement in the hand. The system's software algorithm, when deployed, can detect these “F.R.E.E.” inputs.

The system can measure residual control signals from the user during a set of calibration tasks. A technician, medical provider, therapist or clinical engineer can affix sensors (e.g., EMG, accelerometers, gyroscopes) at numerous sites on the person's body, and other sensors such a video and audio recording, can also be co-registered. A central virtual reality workspace, viewed directly by the user or used as a cue system for the therapist-technician, can demonstrate specific actions and instructions. Instructions can include actually performing or only imagine performing a specific movement or set of movements. The movement can be demonstrated using a virtual reality model, the therapist acting the target movement out, or the participant's own limbs can be moved via powered robotics or stimulation while the participant is instructed to pretend as if he or she were controlling them.

The system can track the signals received from the various sensors and can time stamp them in parallel with registering the time stamps of the various instructions. Once adequate calibration information is acquired (e.g., by having the participant imagine or attempt a variety of functional movements of one or more limbs or the entire body), the technician can trigger the system (or the system can automatically trigger itself) to build mapping models to identify which inputs or combination of inputs correlate with the target activities. This can generate a set of preliminary inputs and mappings and one or more of the instructed activities can be repeated with the preliminary input-motor-output mappings in place. The system can then track the accuracy and speed and query for subjective assessment when appropriate. The software can then update mappings and the cycle can repeat itself, going back and forth between a variety of instructed activities, and various mapping options. Given the combinatorically massive search space of all possible input sources, mappings and motor outputs, to achieve a given functional outcome (e.g., picking up a cup and bringing it to the mouth to drink), a set of mappings and ‘good-enough’ thresholds can be used in parallel.

### Morse-Like Macros

Morse-like Macros can map FREE input to alternative motion mechanisms. For example, instead of using an intact shoulder EMG/mechanical switch to drive distal stim/powered motion, the system can instead map one or more FREE inputs into specific actions (E.g., shoulder shrug once turns on light, twice turns it off). Morse-Like Macros can use any given FREE input in rhythmic manner, with the sequence of intervals to be read-out as a specific assignment, like Morse code, or “shave-and-a-haircut-two-bits” rhythm. The identity of the FREE input (e.g., EMG from proximal weak-side trapezius) and the rhythm (e.g., ..._.._.) can be combined to achieve custom effector outcomes (e.g., map the rhythm to opening a specific phone or computer application, or typing a particular word or phrase). This can leverage the effortlessness and reliability of the human brain to generate intervals between specific discrete actions that can make the engineering decoding aspect of the system significantly faster.

### Neural Graffiti

The system can also implement neural graffiti. When a person imagines speech, a movement or actually speaks or moves, specific brain areas are activated. The goal of neural graffiti is to render the challenge of decoding intended speech or movement commands from activity recorded from the brain much simpler. Decoding neural activity into imagined or intended speech or movement is challenging because the extremely high number of possible speech and language components and infinite potential combinations. Neural graffiti constrains the problem by requesting the user to learn a specific list and set of rules of imagined language-movement ‘gestures’ that can be assembled into intended speech or text, thus replacing the voice activation and typing at a keyboard. The imagined gestures are similar to imagined typing, imagined short-hand (stenography), and imagined sign-language, and can constrain the decoding problem, rendering it tractable and real-time high-speed (namely the intended information can be recorded and decoded at the speed of the thoughts themselves).

There are several ways to approach decoding neural activity into desired text or desired speech:
Imagining speech. One can imagine saying “I wish I could fly” without visualizing the letters.
Imagining typing the text. One can imagine one's fingers typing “I” then “spacebar” then “w” then “i” keys on a keyboard, and so-on, all the while using predictive text, to finish a word and selecting one of the options.
Imagine writing the words as long-hand with a pen or pencil in one's hand, or drawing out the form of each letter using the elbow, shoulder, foot, or even whole body (such as ice skating out the word scratched into the ice).
Imagine writing in short-hand or stenographic terms that indicate the letter-by-letter or sound-by-sound or ideogram-by-ideogram or word-by-word or phrase-by-phrase.
Imagine signing (like sign language with hand/arm gestures) the text letter-by-letter or word-by-word.

The user can imagine a series of swift neural gestures that have features of quick movements and compact notation, such as: Siri as an imagined swirl of the right hand, extending a hand and beckoning with a finger quickly, etc. Options for gestures to spell out letter by letter, word by word, can be stored in a calibrated gesture library.

### Closed-Loop Trigger-Contingent Stimulation

The system can include a “signal source,” a “trigger rule,” and a “trigger outcome.” The “signal source” can be neural activity recorded from the brain, such as scalp EEG, subgaleal EEG, intraosseous EEG, epidural EEG, subdural EEG, intracortical LFPs, depth EEG, single unit recordings, and from other physiological sources such as heart rate, heart rate variability, respiratory rate, galvanic skin conductance, blood sugar level, pupil diameter, extraoculogram, electromyogram, and from other data about the user, such as limb, head or body position inferred from accelerometers, gyroscopes or visual kinematic analysis, and user inputs from the user such as voice, gesture, keyboard entry, mouse click, joystick use, and inputs from other users, such as a physician, teacher, caregiver, and pre-set inputs from the computer, and events pre-defined such as calendar items, email arrival, pre-set timers/tasks, recognition of a particular person via glasses-cameras or ear-microphone voice, occurring, arrival at certain GPS coordinates.

The “trigger rule” can take one or more of these data streams in real-time, in continuous, overlapping or non-overlapping windows of fixed or varying duration, and if a certain set of features in this data were met, such as an oscillatory power feature (such as from subgaleal recorded EEG or the root mean square of EMG over a certain muscle) passing a pre-defined threshold (such as power in a particular frequency band exceeding a predefined number times the standard deviation in a baseline calibration period), then the trigger will be set (e.g., set from 0 to 1), and this can cause a pre-defined “trigger outcome” to occur, with particular outcomes assigned to particular trigger rule events. This outcome may include electrical stimulation at scalp, subgaleal, epidural, intraosseous, subdural, depth, intracortical contacts, muscular FES, and numerous parameters such as which set of two or more contacts (location, spacing, distance, impedance) and the characteristics of the stimulation (duration, pulse width, pulse polarity, pulse shape, pulse isolated or in train, train frequency vs. inter-train interval, amplitude, frequency, phase angle) may be varied. The “trigger outcome” can also be a presentation of certain stimuli (words or images on a screen, vibrotactile patterns, sounds played on a speaker, such as one worn in the ear). For example, a “signal source” might be subgaleal recorded EEG continuously. A “trigger rule” might be an online classifier recognizing that person's unique signature of inattention, poor encoding, confusion, sleepiness, as previously tagged using an earlier calibration algorithm (manual or automatic or hybrid). A “trigger outcome” might be subgaleal stimulation at a certain location, certain power and frequency and certain duration, with or without a spoken instruction into the person's earpiece speaker and a log into the cloud or a worn smart phone device or the device's own data buffer.

### Priming Label Method

This three-step process is designed to enhance encoding, storage and retrieval of episodic memories. ‘Prime’ uses transcranial electrical stimulation (from scalp, subgaleal, or intracranial electrodes), to ‘prime’ the brain in a state ready to learn. ‘Prime’ may occur with an open-loop based on arbitrary onset or state of a psychophysics task, or may be contingent on detection of a ‘trigger signal’ indicating ‘ready to learn.’ ‘Prime’ is thought to be a spatially nonspecific general diffuse modulatory boost. ‘Instruction’ is specific data, for example text or a face-name association or a brief narrative or fact. This can be spoken or displayed. Immediately after delivering instructions there may be a teach back and a ‘label’ such as a vibrotactile pattern or an olfactory cue. The idea is that this ‘label’ can be purposefully voluntarily deployed by the user in the future to help retrieve the ‘instruction’ episodic memory content learned. This approach can be combined with Motor Reinforcement techniques described herein.

### Multi-Modality Feedback

Primary sensory cortex interference stimulation allows two or more conductor contacts to deliver current such that wave interference targets specific targets without affecting intermediate locations. ‘Smart’ refers to taking into account the ongoing endogenous (or steady-state-induced by fluctuating external stimuli) activity, either locally as recorded directly from contacts, or inferred through low resolution tomography inference procedures, such that a lower dose of total current (as a temporal spatial integral, namely amplitude-duration inversely related), were needed to achieve a given targeted electrical stimulation effect. The idea is that electrical stimulation (scalp, subgaleal, intraosseous, epidural, subdural, intracranial, intracerebral, intravascular, transcutaneous peripheral nerve, neuromuscular FES) can induce particular visual/auditory/tactile/proprioceptive alone or mixed or other novel sensory perceptions via delivering stimulation at particular electrodes in a manner contingent on ongoing oscillatory activity: for example if a person were using their Freedom System interface to write a note, subgaleal contacts overlying primary motor cortex, premotor cortex, and possibly parietal planning cortex, or at a particular peripheral nerve, would decode neural activity associated with pre-defined/previously-user-calibrated neural gestures (“neural graffiti”); to allow the user to get feedback to confirm what he or she were gesturing, another set of contacts would generate current (also including mastoid and shoulder and other location reference sites) to induce, for example, visual phosphenes built to form the letters and words, and this could be done contingent on ongoing activity in visual cortex, for example, timing peak of interference stimulation to hit a particular phase of an endogenous oscillation at a particular frequency, for example, 90 degrees on the 10 Hz oscillation. The optimal frequency/phase timing could be explored in a calibration session, and could be updated per day, per week, or continuously as a rolling average or other ongoing self-calibration rule. If the endogenous activity were not enough, the system could purposefully add input, either through an external/semi-external stimulus, for example, flickering light (such as staring at mobile phone screen flickering at 8.3 Hz and then the stimulation interference rides that 8.3 Hz wave, such that images appear on the screen though only seen in brain and not on screen physically out there; or likewise stimulating the median or sural peripheral nerves at 8.3 Hz), though for fully self-contained system, the steady state stimuli may come from tactors embedded in body, such as vibrating motors or speaker diaphragm membranes, or buzzing bracelet, buzzing ring, buzzing earing. The system can combine electrical stimulation at intra- and extra-cranial sites for this cumulative ‘loading.’ Namely, low amplitude electrical stimulation applied across the skin at one or more locations, even when not consciously perceived, could bias sensory cortical activation such that a lower current applied directly to that cortex from the subgaleal electrodes were needed to induce a sensory percept. Beyond fixed steady state frequencies, there could be variable frequencies (random or sweeps) given the computer knows the pattern, it can add interference stimulation to particular phases of that known input fluctuation pattern

A ‘phosphene’ refers to a visual perception of a dot or flash of light in the absence of visual stimulation. Electrical stimulation of the visual areas of the brain will cause a person to perceive a phosphine. This type of ‘induced hallucination’ can also be done for other types of sensation, such as sounds and tactile stimulation. Electrical stimulation of somatosensory cortex may cause sensations of water trickling over the skin, for example, and electrical stimulation of auditory cortex may cause the perception of distinct tones. Electrical stimulation of the vestibular nerve (such as current across the mastoid process), can induce a vivid sensation of the world tilting or turning. Likewise, stimulation of the trigeminal nerve (in the mouth or face), can bias balance perceptions. If higher order cortical areas were stimulated, such as parts of the ventral or lateral temporal lobe, much more elaborate sensations can be induced, such as specific shapes, faces, words, and even re-experiencing of particular remembered events. Henceforth, phosphenes can be considered as indicating any induced experience, whether it be a flash or light or tone or a tactile impression or a balance sensation, etc.

The methods and systems described herein can implement the system to induce specific combinations of phosphenes as a means of feedback from a computer, namely the system seeks to convey images, sounds, tactile and limb position and other percepts, in a manner analogous to looking at a visual display/computer screen, listening to audio, or using a haptic feedback device. The approach can be thought of as a cerebral (mostly via neocortical stimulation, though the “temporal interference” and “short intersectional pulse” techniques will allow sensory thalamic/LGN/MGN/VP and other subcortical activation) equivalent of what epiretinal, cochlear and auditory brainstem implants do. Of note, the induced percepts may not require the same spatiotemporal resolution as might be required, for example, of a cochlear implant, because they will be part of a closed-loop system that the person will learn to interpret; hence phosphene-based visual and auditory feedback does not necessarily need to create a vivid 3D image or sound, and in fact creating too vivid a percept would likely be counterproductive as it could interfere with ongoing experience of actual, real visual, auditory and other sensory input. Instead, the induced-phosphene-feedback system is intended as a milder, non-distracting overlay that conveys sufficient information to make the closed-loop system useful, such as allowing the user to know they have successfully entered data, or to read back text they just entered.

Phosphene-conveyed information may include:
Spatial orientation cues, such as a map route overlaid on the scene, or the framework of a building and locations of internal wiring/pipes, or diagnostic information overlaid on the patient a physician were looking at, stress fractures in a material an engineer were holding, radio/X-ray/infrared data in the sky that an astronomer were viewing, etc., a kind of ‘hallucinated’ ‘augmented reality’ to see things that would be invisible to the naked eye/ear/hand etc.
‘Visual’ text, ‘auditory’ speech, ‘tactile’ impressions of language, such that if there were text on a computer screen or smart phone, rather than displaying it visually on the screen, or playing it as audio, it would be delivered via this phosphene-percept system, this could include ideograms/idiophones, akin to Emoji's, hieroglyphics, icons, sign-language gestures, Chinese characters, etc., namely specific visual/auditory/spatial patterns that could be unpacked and interpreted by the user in a more compact manner, both at word-by-word and grammatical and narrative levels, so the symbol ‘&’ to mean ‘and’ and an arc shape in musical notation to group a phrase or an indentation for a paragraph
Images and sounds, tactile sensations, tastes, smells, balance perceptions
For somatosensory phosphenes it is important to note that the same electrode arrays that can be used to deliver current into somatosensory cortex and ventroposterior thalamus can also pass current to scalp cutaneous nerves and these can be used additively to induce percepts

### Motor Reinforcement

A key innovation of the Freedom System is that it can combine output effectors in a variety of ways. Thus, a given output effector may be used to achieve a specific actual functional movement, or may be used to enhance motivation, plasticity, arousal such that the user is able to learn more quickly and effectively how to deploy the Freedom System or its components for particular tasks.

Electrically conductive contacts placed on the scalp, the earlobes (passing current over the auricular branch of the vagus), perispinally (epidural spinal root stimulation implanted or transcutaneously along the vertebra on the skin of the back), the periphery (such as the median nerve at the wrist or the sural nerve at the ankle), or implanted (such as in the subgaleal, intraosseous, subdural, intracortical or depth locations) can be used to bias activation of the brainstem, sensory pathways or motor cortical areas themselves. Anodal (positive, excitatory) stimulation can be timed to coincide with the instruction to imagine, attempt or perform a specific action (e.g., wrist flexion or picking up a pen) and then later during free-running daily function; there is compelling data that anodal stimulation to primary motor cortex enhances post-stroke functional movement and this is expected to apply for other neurological conditions as well and other anatomical targets (premotor cortex, supplementary motor cortex, ventrolateral thalamus, cerebellum including deep cerebellar nuclei). Unlike trials in which electrical stimulation is applied in a tonic manner during physical therapy, the Freedom System can trigger electrical stimulation at one or more sites (scalp, brain, peripheral nerve etc.) at precise times in a given functional movement or task, for example upon flexion initiation, or in the bring-to-mouth phase of bringing an object to the mouth, or a particular stance in a gait cycle or wheel position in a bicycle. Likewise, the Freedom System may dynamically adjust the stimulation parameters (source and sink contacts, frequency, amplitude, etc.) at different phases of particular functions and movements.

### Buffer-Feedback

Neural gestures to enter data, decoded real-time, stored in buffer in chip either in the body or worn on the body (e.g. behind ear or on glasses or wrist), or to laptop or phone or other receivers nearby. Feedback can include audio to a speaker in ear, images on a screen/display, and most importantly tactile as electrical stimulation patterns via subgaleal leads (including interference patterns to sensory cortex to visual cortex to auditory cortex, as visual/auditory/haptic ‘phosphenes’).

### Frequency Tagging Reinforcement

Frequency tagging is defined as a technique whereby an input, such as visual (text on a screen, particular images), audio (such as spoken word, music, tones), and tactile sensation (tactors vibrating) are set to fluctuate at particular frequencies such that they induce a steady state evoked potential in the brain, such that neural and muscular recordings (such as from scalp EEG, subgaleal EEG, intraosseous EEG, epidural EEG, subdural EEG, depth EEG, micro-LFP, single unit recordings; surface EMG, implanted EMG) can detect these frequencies (such as from Fast Fourier transform, wavelet convolution, Hilbert transform power and phase) to infer the presence, timing and location of processing of the fluctuating stimuli. The premise of this electrical stimulation interference method is that electrical stimulation (whether delivered non-invasively from the scalp through tDCS/tACS/tSOS, remotely rTMS, or current delivered by subgaleal, intraosseous, subdural, depth, intracortical contacts; or to peripheral nerve, perispinally, spinal epidurally, skin EMG, implanted perimuscularly) at the time that frequency tagged stimuli are presented will selectively reinforce circuits and synapses related to those stimuli. The electrical stimulation linkage to the frequency tagged stimuli could be based on simple coincident timing (i.e., stimulate brain electrically when fluctuating stimuli presented), and more effectively when the precise timing and amplitude characteristics of the electrical stimulation match one or more parameters of the stimulus frequency (e.g., tDCS at 3.26 Hz at the same time as a visual image flickering at 3.26 Hz). This can be accomplished by gross timing, or by precise phase locking (e.g., the phase of the 3.26 Hz electrical oscillation matches the phase of the external stimulus 3.26 oscillation, or, the relationship between the two has a precise phase offset, such as 45, 90, or 180 degrees, or uses a phase precession sliding phase relationship), and where there could be a fixed non-equal relationship between an external stimulus frequency and electrical stimulation frequency (such as audio fluctuation at 2 Hz and electrical current oscillation at 4 Hz), using harmonics and other frequency relationships, and where the underlying frequency of both the external stimulus and the electrical stimulation can also vary dynamically (i.e., zap frequency sweeps, noise-like pseudo-random), and where both the stimulus frequency and electrical stimulation can be contingent on underlying endogenous signals (such as immediately preceding EEG signatures, and other physiological metrics such as EKG, HRV, GSR, and other inputs such as body/joint limb position). The frequency-tagging-stimulation-interference algorithm could also use phase-amplitude coupling, such that the frequency of electrical stimulation could depend on the amplitude of the external stimulus fluctuation, or the amplitude of the electrical stimulation could depend on the frequency of the external stimulation. If we define oscillation feature as any parameter of an oscillation that can be set or observed, such an oscillation's polarity, waveform shape, anatomical location of the oscillation known or inferred (e.g. via LORETA), traveling wave direction, presence/absence, timing onset/offset, the power in a particular frequency, the frequency where the peak power were, the phase of oscillations band-passed any particular frequency, then we can assert a contingency rule where any particular oscillation feature of the external stimulation (flickering stimulus) or any particular oscillation feature of the electrical stimulation is contingent on a particular oscillation feature of neural activity recorded at one or more locations. For example, upon detection of an increase in power above a pre-defined threshold (such as two standard deviations above a baseline recording from that region) of 3 to 4 Hz activity recorded from two or more contacts, this may trigger high frequency electrical stimulation at those same or another set of contacts, or may trigger a distinct frequency or power of external stimulation oscillation. More elaborate triggers (to be used in contingency rules) may include measures of inter-contact coherence, phase alignment, mutual information, and phase-amplitude coupling. In addition to externally-driven steady state potentials, the system can time electrical (or optical) stimulation to be phase aligned with existing endogenous oscillations in particular frequency bands, at particular phases.

### Cortimo Operational Principles

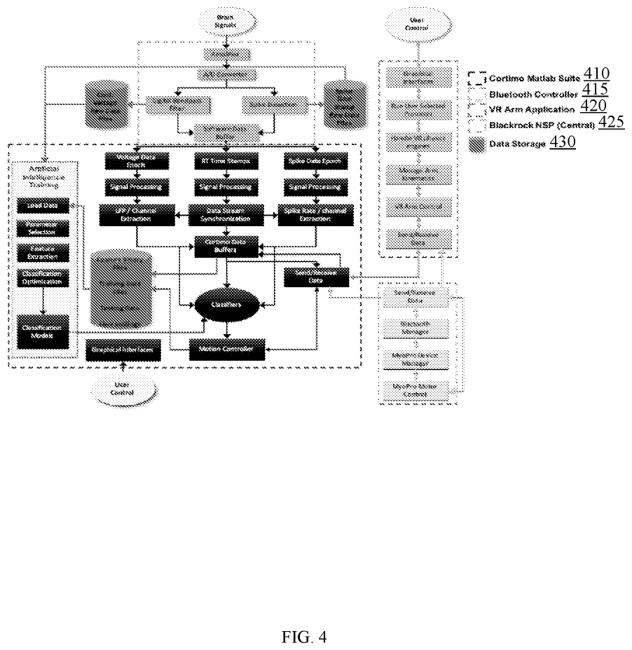

FIG. 4 depicts an exemplary embodiment of the system (Cortimo Software Suite) for motor function facilitation according to the claimed invention. The system depicted in FIG. 4 can be an example of the motor function facilitation system in FIG. 2, and can emphasize the operational principles of the Cortimo Software Suite and how the several software components are integrated. Briefly, brain signals are recorded, amplified, pre-filtered and digitized using a Neural Signal Processor (NSP) 425. Additionally, Central software allows for functionalities such as signal processing, data filtering, spike detection and data storage. The NSP 425 performs real-time data streaming to a dedicated UDP port, where the data are stored in data storage 430 (e.g., buffer) that can be accessed by the Cortimo Suite using APIs.

The Cortimo Suite can read brain signals from the data buffer at user selected time intervals (ranging from 50 ms to 100 ms). Once the data have been queried from the buffer, the Cortimo suite runs dedicated Brain Computer Interface signal processing and decoding algorithms to provide brain derived control for the external applications. The first step of the algorithm is to prepare and unify the raw voltage channels and spike time stamps derived from the NSP, and get the data streams ready for further processing. At this stage, the Cortimo Suite uses time stamps derived from the high-precision NSP clock to synchronize the data streams and the various software components.

The Cortimo system is designed to exploit two main brain signal components: spike count and Local Field Potentials (LFPs). Following data synchronization, the signals get divided in user defined time windows and processed to reduce noise and artifacts before performing feature extraction. Subsequently, depending on user-selected analysis parameters, the two feature streams and the real-time Cortimo data are saved to dedicated files for optional offline analysis and machine learning training.

Real-time feature extraction is a paramount step in the algorithm, because the extracted features are then fed into the classification models (decoders) for decoding brain intent and thus, choosing the most discriminative features will greatly benefit Brain-Computer Interface (BCI) decoding performance. The decoding output is then converted into a motor control command and sent to the VR Arm application and the Bluetooth Controller via the internal UDP communication layer. The Cortimo decoding approach is designed to be flexible and take fully advantage of the available training data and current BCI performance. In the past, BCI applications, despite allowing for parameter re-training and adaptive feedback loop, have been designed for the optimal implementation of a single predetermined decoder (e.g., a classification method). Cortimo Suite implements a different solution, in fact the system provides a set of seven different classification models ready to be deployed after a brief training data collection. In other words, Cortimo allows the operator to identify, select and test different decoding approaches with just a few interactions with the graphical interface. This design choice addresses one of the main limitations with current BCI systems, namely the lack of performance generalizability of BCI systems across different sessions and subjects. Cortimo decoders can be quickly trained, using embedded offline tools that are capable of using either the raw data files or Cortimo-generated feature binary files to run a fast optimization procedure to identify the best features and the best-performing classification models for the specific dataset used for training. Furthermore, these models and their parameters can be stored in files and easily loaded into the Cortimo Suite for real-time use.

### Training/Testing Protocols

The Cortimo Suite provides a full set of training and testing protocols exploiting the functionality of the VR Arm application and its integration with the real-time processing. In addition to providing a realistic model of arm kinematics, patient visual feedback and motion targets for practice and rehabilitation, the VR Arm application collects crucial real-time information regarding that are relayed back to the Cortimo software for file storage. These files provide fundamental data, such as timing information, data labeling and arm kinematics that are used for the offline machine learning methods of the Cortimo classification models. Moreover, the VR Arm application represents a valuable tool for designing and administering innovative rehabilitation protocols that can be fully automated and instrumented to ensure optimal training and patient compliance with the therapy.

### Closing the Feedback Loop

The Cortimo Suite aims to achieve optimal real-time use and performance as opposed to most common BCI systems, whose performance is evaluated using offline tools. Thus, the system capability of providing accurate real-time feedback and minimum latency is important. The Cortimo system has been designed to ensure that all the relevant feedback information gets updated at each computational cycle. This has been achieved using the streaming capabilities of the wearable device and integrating the wearable device backend in the Bluetooth Controller Software Module. This module provides a stable and consistent communication channel between the wearable device and the other components of the Cortimo Suite, thus creating a reliable closed loop system. In addition, the Bluetooth Controller Software Module implements the control logic for the wearable device motors, offering two different control strategies: At regular time intervals of 100 ms, move the motors in a specific direction (flexion/extension), based on the patient's brain intent to move their arms and hands, thereby implementing a discrete control strategy; and Using the patient-derived signals to send the motors to a specific position, expressed in terms of joint angles, with a single command, thereby implementing a continuous control strategy.

### Cortimo Matlab Suite

The real-time Cortimo Matlab Suite 410 can collect data from the Multiport Central software, using proprietary API. The API and the Cortimo Application have been integrated using the Matlab programming environment. However, in some cases, the API and Cortimo Application can be integrated using other programming environments such as Python- or C++-based environments. The Cortimo Suite can also synchronize and integrate the neural data and the classification output with kinematic data derived from the wearable device joint motors. This bidirectional communication is implemented using asynchronous UDP local-host datagrams that are exchanged between the Matlab tool and the C++ Bluetooth Controller Software Module (BCSM). After deriving brain-derived arm controls, the Cortimo Application will send the output to the Bluetooth Controller Software that will provide an interface to control the wearable device and simultaneously receive feedback from it.

### Bluetooth Controller Software Module (BCSM)

The Bluetooth Controller Software Module (BCSM) 415 implements a bidirectional communication with the Cortimo software, using local-host asynchronous UDP communication and also a bidirectional communication, via Bluetooth, with the wearable device firmware running on the integrated circuit mounted on the powered brace. In more detail, the Bluetooth Controller Software Module (BCSM) is responsible for:
managing the Bluetooth communication with the wearable device firmware;
generating real-time commands for the wearable device firmware;
receiving and managing real-time feedback from the wearable device, such as motor position, battery life, EMG readings, range of motion limits; and
when necessary, adjusting the wearable device settings.
   The interface between the Bluetooth Controller software and the wearable device is implemented using (proprietary) wearable device APIs developed in the C++ programming language.

### Virtual Reality Three-Dimensional Arm Application

The VR arm 420 is a stand-alone Windows application that implements a realistic and physically accurate arm and hand model. The model has been developed to mimic the behavior and controls of the wearable device and it can be controlled using similar input commands. Furthermore, the VR application can be used to display in real-time the kinematics of the wearable device, thus providing a valuable tool for visual feedback. Another important aspect of the VR application is its capability of implementing training and rehabilitation protocols for use with and without the wearable device. The application has a series of GUIs that can be used to set up specific training protocols for automating the rehabilitation sessions. For instance, the operator can choose to display different types of motion or specific target arm positions to guide the subject during the session. The VR arm is also capable of collecting data and parameters of the BCI and rehabilitation sessions. This information is relayed in real-time back to the Cortimo Matlab software and used to close the feedback loop between the system and the subject. The VR application exchanges real-time data with both the Cortimo Matlab Suite and the Bluetooth Controller Software Modules using local-host UDP communication protocols on dedicated and secure ports.

### Cortimo Suite Graphical User Interfaces (GUIs)

In this section the main Graphical User Interfaces (GUIs) that represent an important aspect of the Cortimo Software are introduced. It is essential to highlight that the GUIs have been designed to allow users to easily, intuitively and with minimum training manage and run comprehensive Cortimo BCI sessions. The GUIs allow for complete customization and changes in the analysis and protocols, without requiring programming skills and code modifications. In other words, the code behind the GUIs will handle all the parameters allowing the Cortimo to provide unique user-friendly functionality and flexibility. In fact, the user can easily choose the most appropriate settings and change them at any time, without compromising the system behavior or performance. Moreover, the GUIs will provide context driven feedback at each step to help guide the software stakeholders in setting up optimal BCI sessions. The Cortimo Suite deploys several GUIs that can be managed simultaneously and control all the BCI analysis and settings. In addition, the Cortimo Suite provides patient specific views, that only display simplified visual feedback for the patient at runtime. These patient views differ from the operator GUIs, where all the relevant analysis parameters and settings are also displayed and modified.

### Main GUI

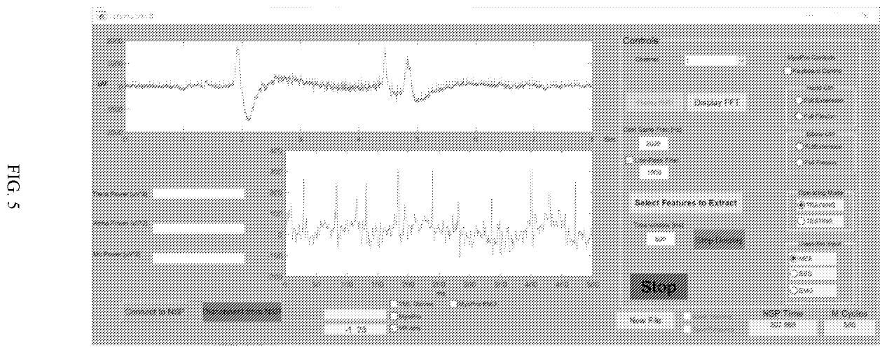

The main GUI provides all the controls necessary to manage the BCI session, connect the main software components, manage the training and testing protocols and display all the necessary information. FIG. 5 depicts the main Cortimo GUI.

### BCI Analysis Parameter Selection GUI

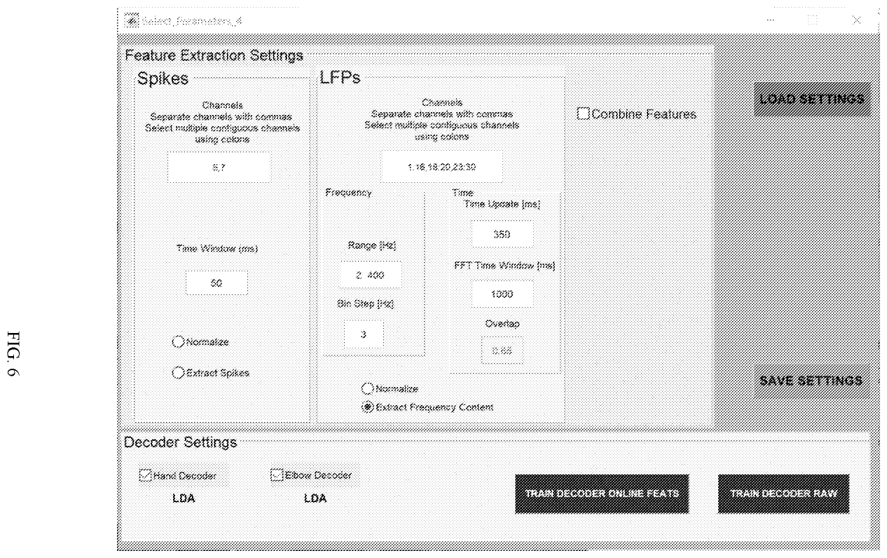

The parameter selection GUI can be accessed directly from the main GUI and provides all the functionalities necessary to select and manage the BCI analysis parameters, the feature extraction and the decoding techniques. Furthermore, the parameter selection GUI allows the operator to easily train, re-train and load pre-trained decoding models. Using this GUI, new decoding approaches can be created and directly imported into the real-time Cortimo operations. The parameter selection GUI is depicted in FIG. 6.

### FFT Display GUI

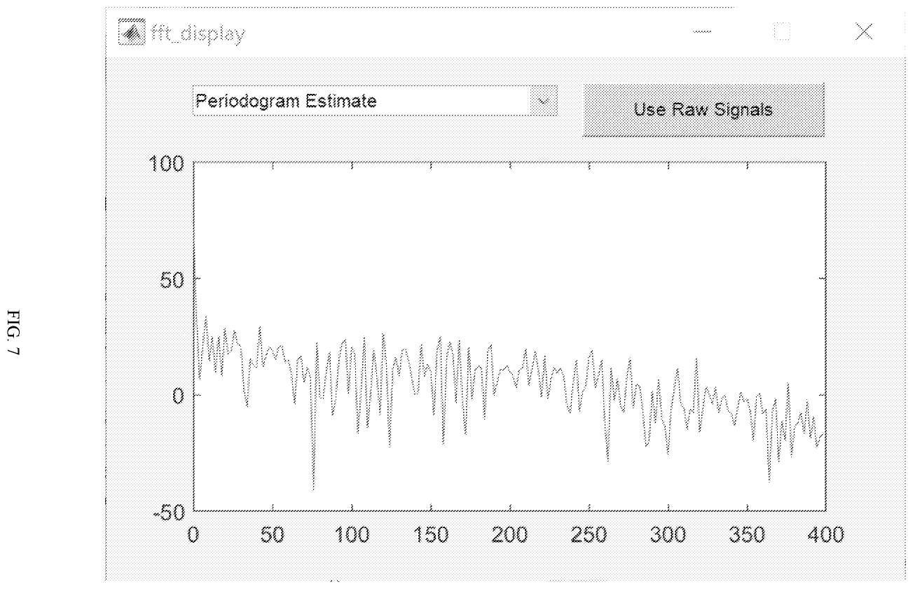

The FFT GUI can include a real-time display of acquired signals. The FFT GUI can allow for a quick and immediate assessment of the acquisition system performance and signal properties. The FFT window can also be used for a quick assessment of the Cortimo input filters and their performance. FIG. 7 depicts a the FFT GUI.

### Cortimo VR Arm GUIs

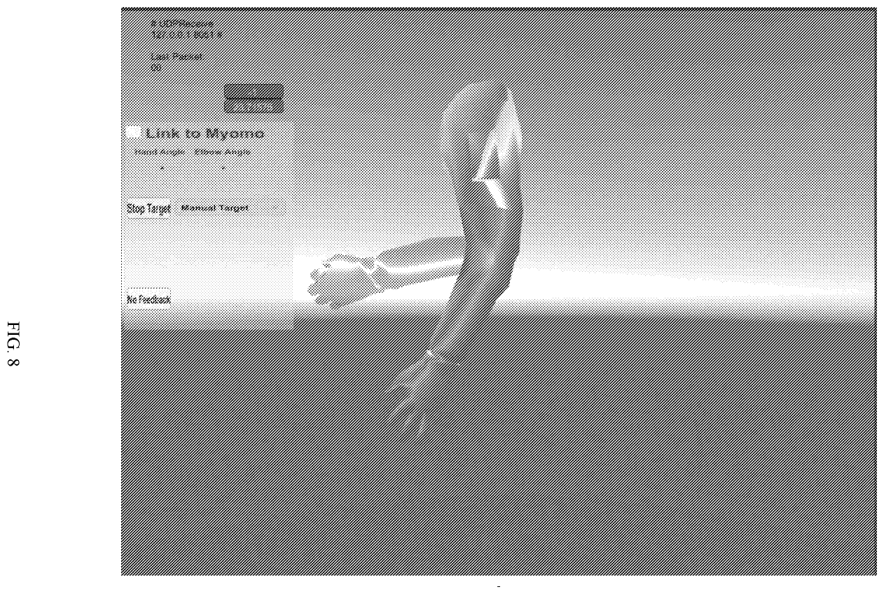

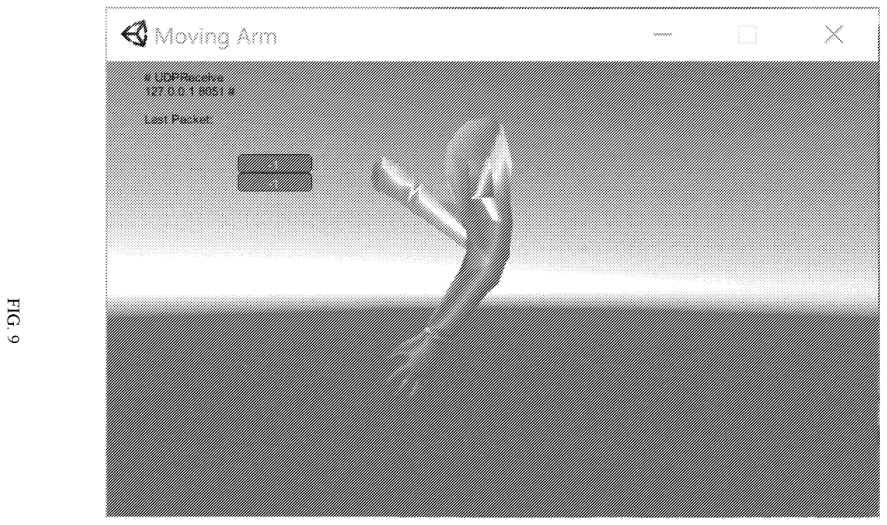

The Cortimo VR Arm Application can include two GUIs that are displayed on two separate screens, one for the system operator and one for the patient. FIGS. 8 and 9 depict the VR Arm Operator GUI.

### Operator View

The Operator View compile all the relevant information of the VR app and the training/testing and rehabilitation protocols to be administered to the patient. This user-friendly GUI allows for quick set up and management of all the delivered protocols.

### Patient View

The Patient View display a simplified version of the Operator View. This GUI can display visual feedback for the patient during BCI sessions. Application controls and additional information can be omitted to allow the patient to completely focus on the movements of either the VR arm or the wearable device.

### System Behavior

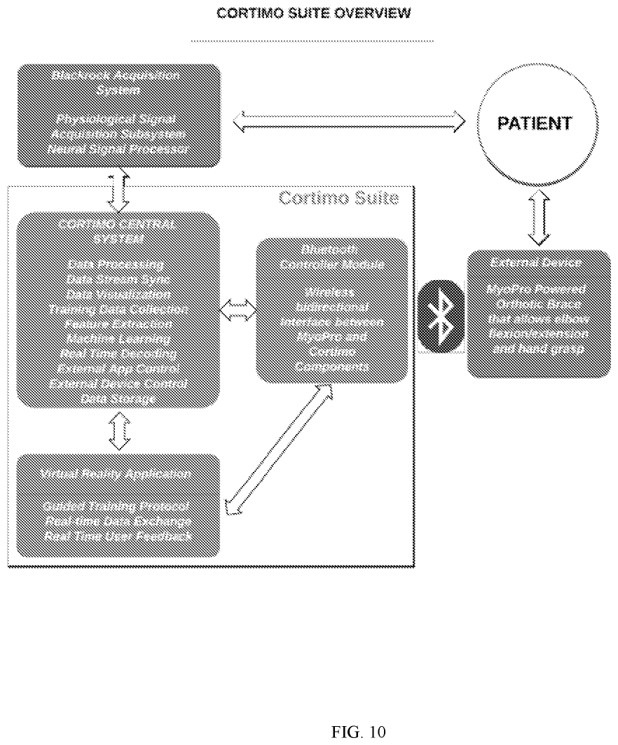

The system behavior overview can both identify the main system components and their interactions and validate the system outputs. The high-level overview shown in FIG. 10 will be followed by a more specific Cortimo architecture description.

The block diagram in FIG. 10 presents the main components necessary to the Cortimo BCI Suite. Brain signals can be collected using the Neuroport acquisition system. After signal pre-processing and digitization, the resulting digital voltage channels and spike count data can be collected by the Cortimo Central Application via a User Datagram Protocol (UDP) connection and dedicated APIs. The Cortimo Suite can be the BCI system central hub and can perform advanced real-time signal processing and feature extraction. Subsequently, the extracted features can be fed into a patient-specific decoder that can be quickly re-trained taking advantage of newly available data sets. The decoder can translate the patient's brain signals into user's movement intention and transfers these commands to the wearable device, e.g., the MyoPro, via the Bluetooth Controller Software Module (BCSM). The BCSM manages the bidirectional wireless communication between the PC applications and the firmware running on the powered brace. This communication protocol can be specifically designed for the Cortimo BCI application and utilizes proprietary APIs. Finally, the Virtual Reality application can exchange data with other entities in the Cortimo Suite for both testing and training the system. The VR application can also include a realistic visual interface between the patient and the BCI operation providing real-time feedback of arm movements and rehabilitation protocols.

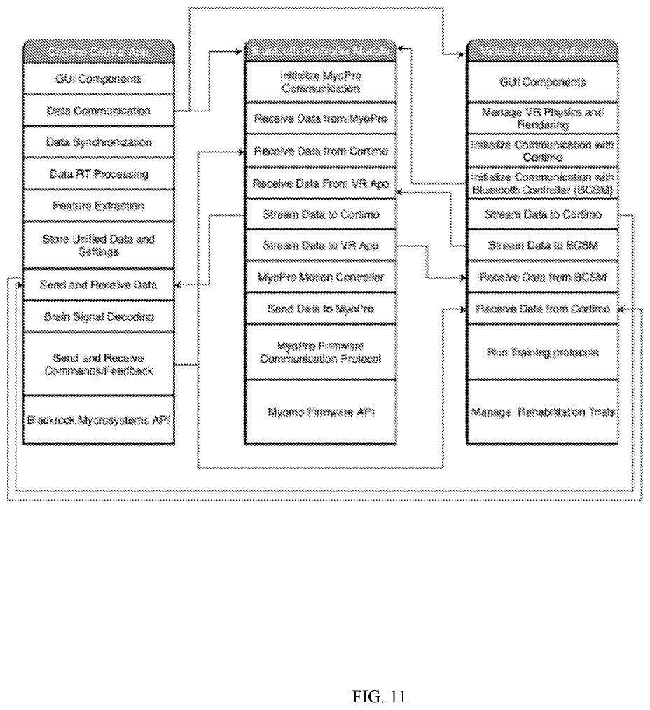

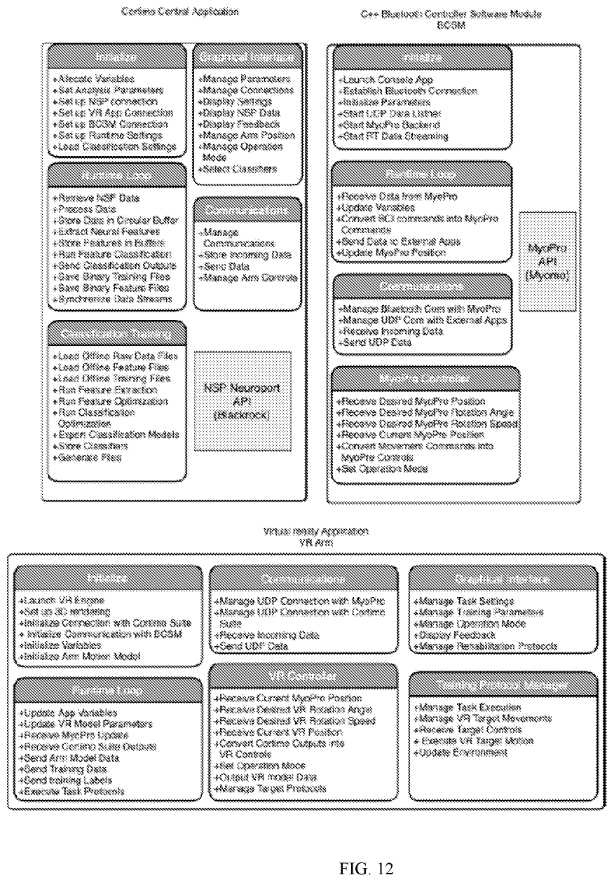

FIG. 11 depicts a high-level design of the Cortimo Suite and its main components. The three main software modules are further broken up in the major logical attributes that define their behavior. FIG. 12 depicts a mid-level Cortimo application architecture. Further. the three major software components of the Cortimo Suite can operate asynchronously to maximize hardware/software performance at run time. The different data streams and data communication between modules can be performed asynchronously. Thus, the Cortimo application can act as a central hub and perform data synchronization. Each system component can generate and stream data at different speeds. These data and their corresponding sampling rates can be stored in dedicated low-level hardware/software circular buffers. Then at regular time intervals, the Cortimo application cam query all the low-level buffers, processes all the available data streams and can store them in dedicated high-level software buffers. Subsequently, the data streams can be unified using the NSP absolute time stamps and finally stored in binary data files for optional offline analysis. This approach can rely on the absolute time stamps that are derived from the high precision clock of the Neural Signal Processor.

### Cortimo Application

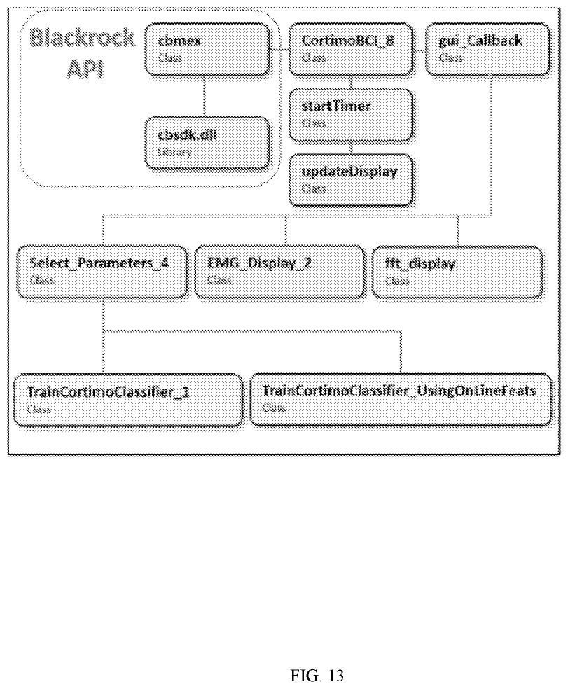

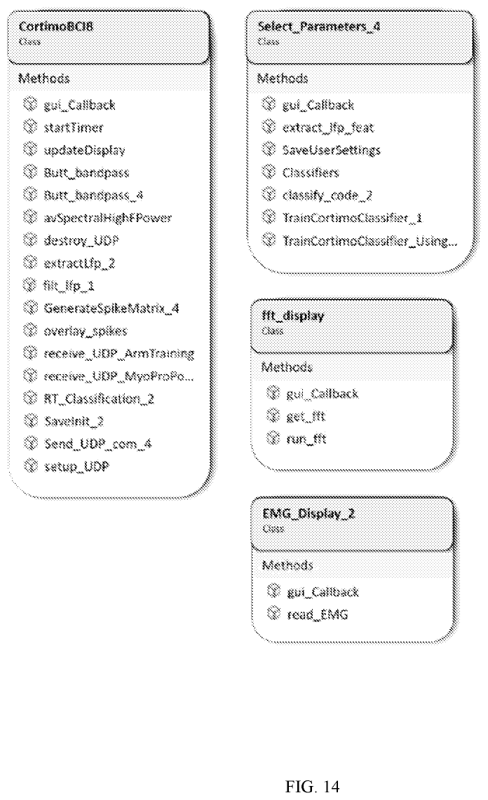

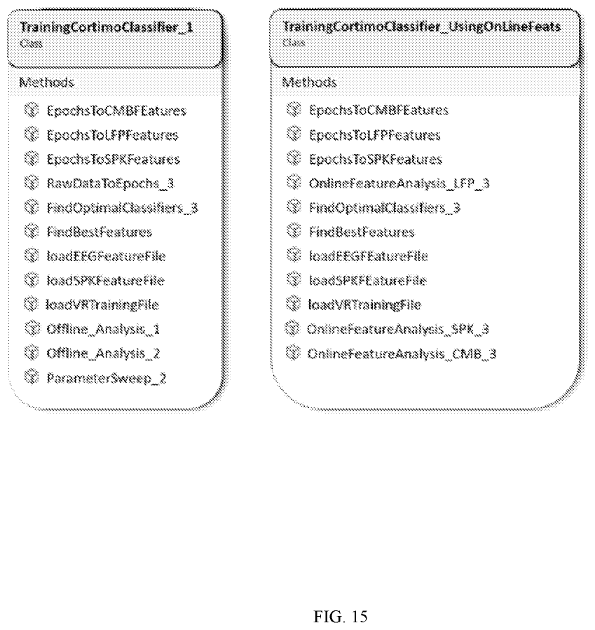

FIG. 13 depicts the Cortimo Application class diagram. The Cortimo Application can read the NSP data buffer in pseudo-real time. This API can include a binary library, the cbsdk.dll, Matlab specific mex files, and cbmex files. The main class can be the CortimoBCI_8 that runs the code behind the main GUI and the main Cortimo functionalities using the startTimer class for setting up the major parameters, and the updateDisplay class that can represent the main application loop that is triggered at user-defined time intervals (e.g., ranging between 50 ms and 100 ms). The updateDisplay can define the real-time behavior of the software, and can perform the brain signal processing and decoding. The Cortimo Suite can be the core of the BCI system and can also feature additional code for managing the additional GUIs and data communication, synchronization and storage. Another important component of the Cortimo Suite can be the offline training capability for the classification models defined in the classifiers class. Namely, the TrainingCortimoClassifier_1 and TrainingCortimoClassifier_UsingOnLineFeats classes can perform all the necessary steps to perform machine learning training and can generate new decoders and settings. These classes can implement two important but different training strategies. The former class can allow the user to select brain raw data to be used for feature and classification optimization, while the latter can allow the user to select Cortimo binary files containing features that have been extracted online to be used for classification optimization. In other words, the latter approach can utilize features that have already been extracted (in real-time), while the former approach can allow for selection and extraction of new features. FIGS. 14 and 15 depict the main classes for the Cortimo Suite functionality during the runtime execution.

### Bluetooth Controller Software Module

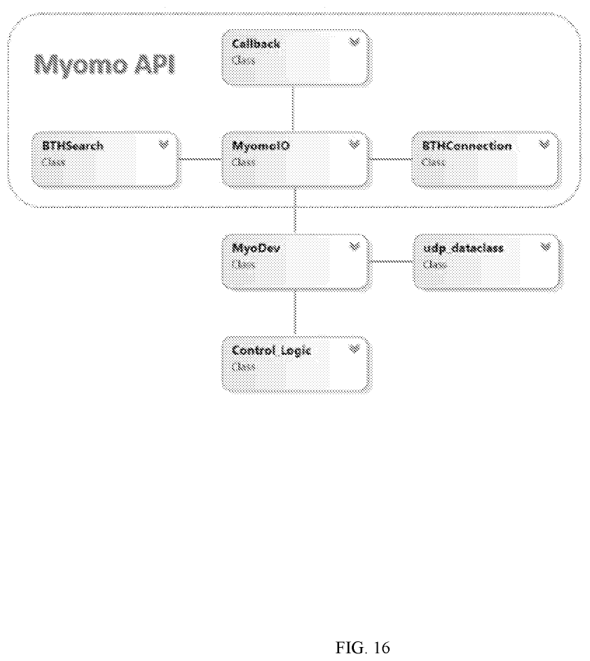

The Bluetooth Controller Software Module can be a stand-alone application that runs in the background and allows the Cortimo Suite to manage and communicate with the wearable device by talking directly with the firmware embedded on the device, using native command strings that are defined in the manufacturer API. FIG. 16 depicts a main class diagram for the Bluetooth Controller Module.

The main class to interact with the wearable device firmware is the MyomoIO, which can act as software gateway for bidirectional Bluetooth communication with the device.

The main application class is the MyoDev. In this class the application main loop can be defined and regular callbacks to all the other classes (control and communication) can be executed. It is within this class that the Bluetooth Controller Software Module can gain access to the wearable device backend. The Control_Logic class handles the control strategies for the wearable device, receives input commands from the Cortimo Suite and translates them into command strings that the wearable device firmware can interpret and execute. To ensure patient safety and comply with the FDA required safety mitigation factors, the Cortimo communication with the wearable device can implement commands that are deemed to be safe by the wearable device API, the system is designed to ignore wrong or faulty commands and can operate within the wearable device manufactured-prescribed indication of use. Furthermore, the Control_Logic class can implement different types of motor control strategies and can convert wearable device backend calls into real-time kinematic feedback information that can be consumed by the Cortimo Suite.

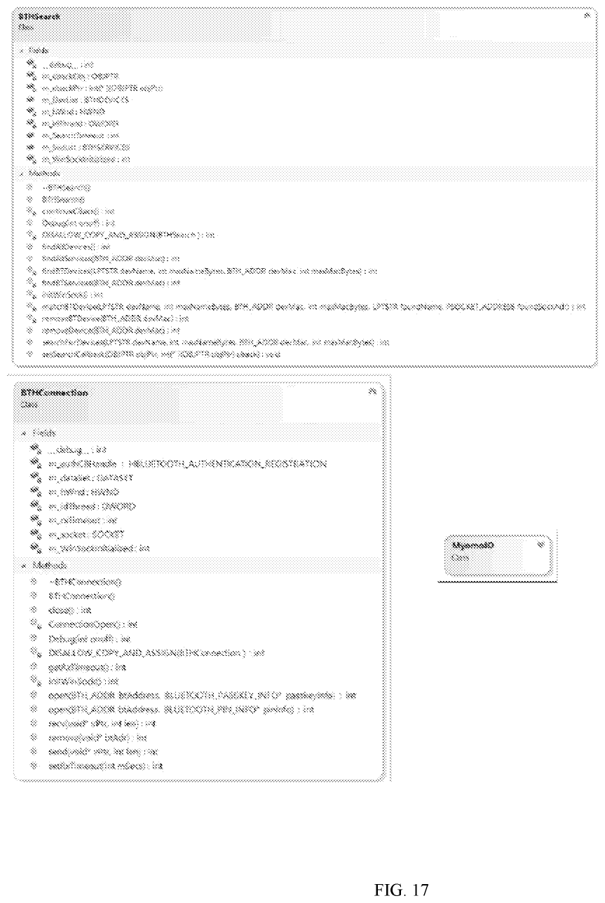

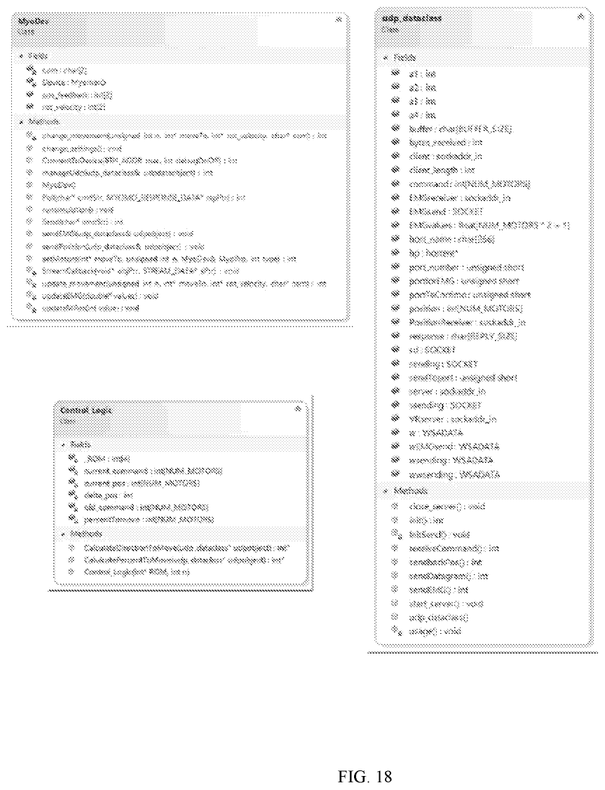

The udp_dataclass class can implement bidirectional communications with the other Cortimo software components. This class can execute asynchronously and exploit multi-threaded processes for performance optimization. FIGS. 17 and 18 depict main classes for managing the behavior of the Cortimo software components. FIG. 17 depicts main classes handling the communication protocol for the wearable device, and FIG. 18 depicts main classes managing and controlling in real-time the wearable device.

### Virtual Reality Arm Application

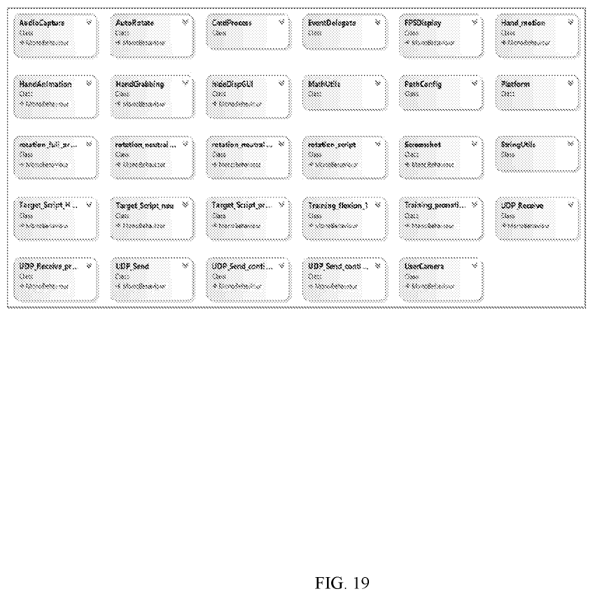

The VR Arm Application is a multi-threaded application that deploys the latest-generation Unity 3D engine capabilities to handle the virtual arm kinematics and physics in real-time. The application consists of several classes that run in parallel and are executed concomitantly at regular time intervals. The main classes are depicted in FIG. 19 and their fields and methods are expanded in the following figures.

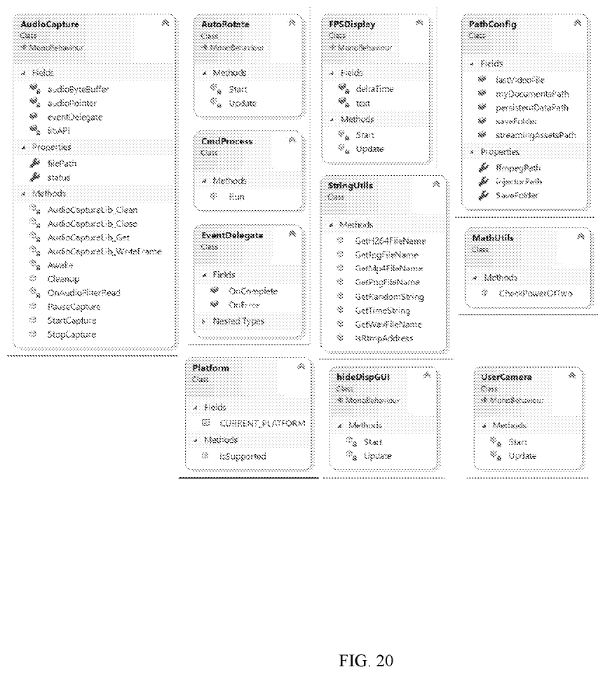

FIG. 20 depicts the main classes that are responsible for handling the VR arm application graphics, properties, behavior and performance. These classes are typically found in Unity3D engine applications, although they can be auxiliary to the execution of the arm application. These classes can form the application framework and handle runtime events and interrupts.

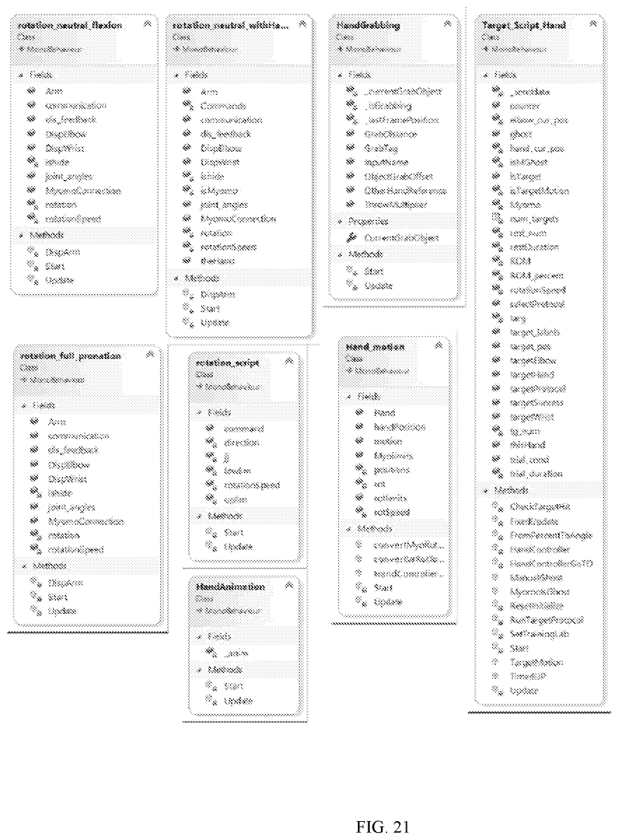

FIG. 21 depicts the main classes controlling behavior and properties of the virtual arm and hand in the application. For instance, the rotation script class can perform all operations to create a bidirectional interface between movement commands and virtual arm/hand controls. The movement commands can be received either from operator's inputs, such as GUI commands or keyboard keys, or from the output of the Cortimo Suite motor control layer. Once the movement commands have been interpreted, the commands can be applied to the 3D physically accurate arm model by specific rotation/hand movement classes. The actual classes that are used for the VR motion can depend on the operator choices, protocol and type of motion that can be selected at runtime.

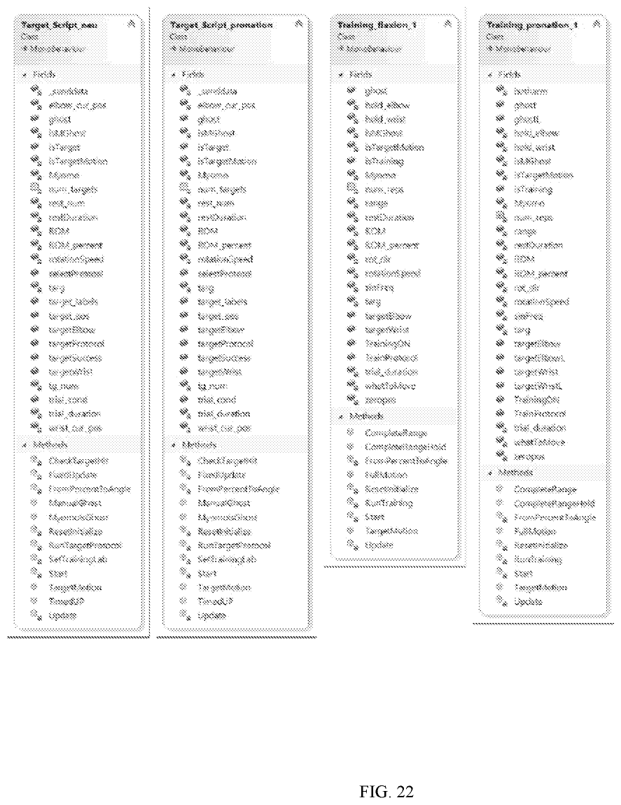

FIG. 22 depicts main classes managing the training and testing protocol for the VR application. The Target_Script classes can implement and manage the training and rehabilitation protocols. The operator can choose between several types of protocols using a dedicated GUI and the Target_Script classes take care of their execution, timing, and feedback data collection.

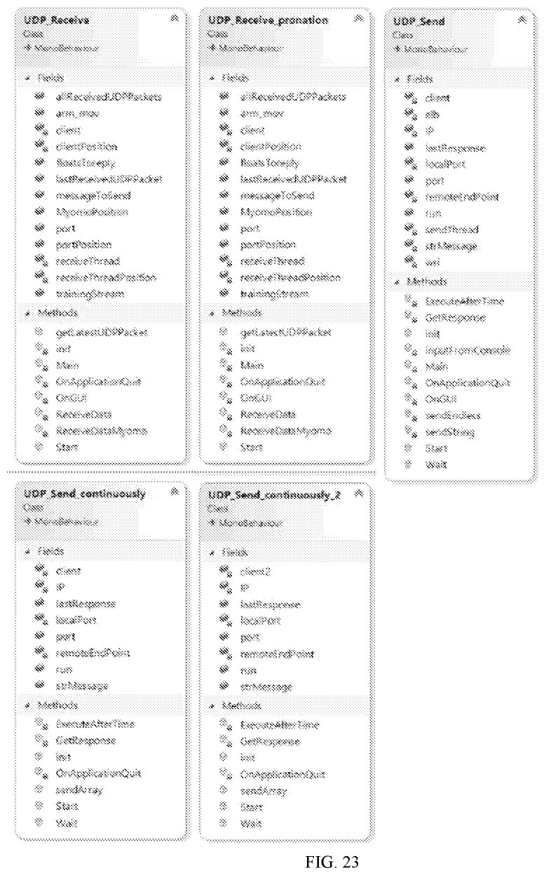

FIG. 23 depicts the classes that are deployed to handle the multiple real-time bidirectional UDP communication channels with the other Cortimo software components. The UDP communications can occur asynchronously and that the VR Arm application can run independently of the presence of a wearable device. In other words, the VR application can be a stand-alone software that can be used combined with brain derived or other control signals for training and testing of both system and subject's performance. For instance, a new decoding approach or parameter set can be tested without the need of donning (or using) the external powered brace. Furthermore, it is worth noting that the VR arm application is a valuable and unique tool to easily and automatically collect fundamental training data for the machine learning algorithms upon which the Cortimo decoding approaches rely.

### Application Threads

As previously mentioned, the Cortimo can include three multi-process and multi-threaded main applications. These components can operate asynchronously, maintaining regular and unmanaged communication channels that allow for almost real-time data exchange. The components can run independently and rely upon separate main threads. This compartmentalized approach allows the Cortimo system to handle runtime issues using a series of warning messages which can achieve two objectives. First, the Cortimo system can efficiently handle internal crashes, without freezing the system nor halting the BCI experimental sessions. In case of a component malfunction, the system can isolate the problem and attempt to resolve it. In case additional inputs from the system operator are needed, the system can communicate very specific instructions using clear and easy to understand graphical elements. Second, the main Cortimo system can recover from multiple component issues while ensuring patient safety or data integrity.

### Connection Threads

The Cortimo system can also include a series of connection threads. The connection threads can ensure real-time asynchronous communications between the components with minimum system latency and workload. The connection threads run in the background and provide real-time data exchange capability.

### Physical View

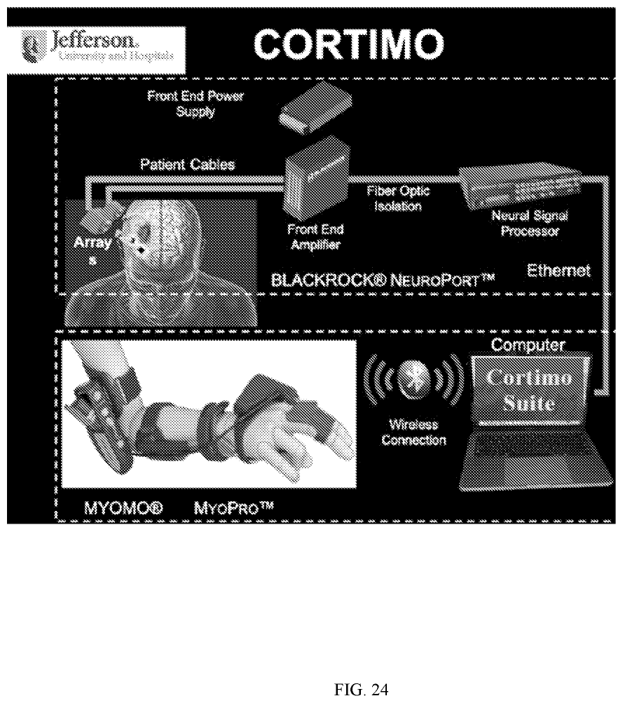

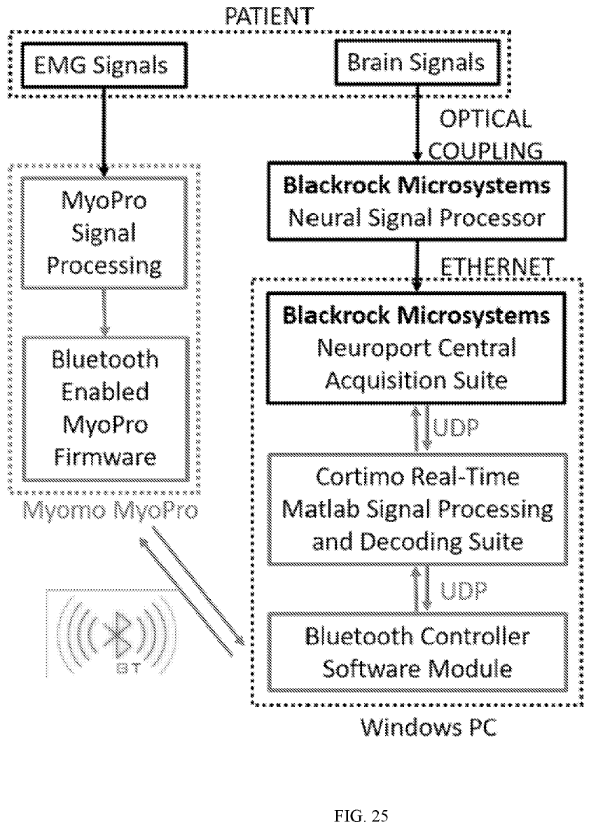

The Physical View can present the Cortimo system hardware components on which the software modules are executed. This view is helpful to highlight the physical connectivity between the components. FIG. 24 depicts a physical layer of the Cortimo Suite with the system main components. FIG. 25 depicts a block diagram of the Cortimo system, including software components and inter-component connectivity.

### Use Case View

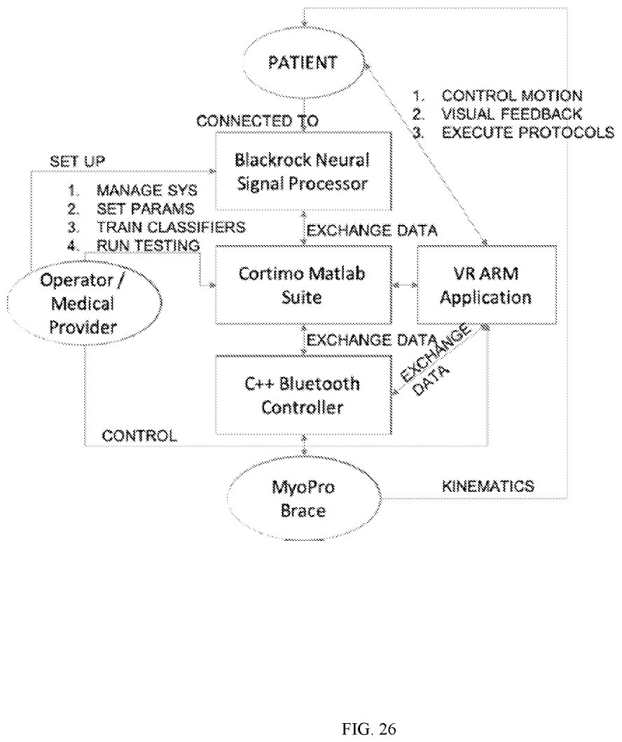

The Use Case View can illustrate the user's actions on the system components. FIG. 26 depicts communication routes between actors and entities involved in a Cortimo BCI session.

### Cortimo Component Communication Protocol

As mentioned in the above sections, the Cortimo components internal communication can be based on the UDP protocol. Each Cortimo software component can act as a UDP listener and sender independently of all the other software processes and carry out data exchange with high temporal precision and minimum latency. The use of both low-level and high-level data buffers combined with precise time stamps and metadata can ensure data integrity even in case of latency due to hardware or software workload. Furthermore, the use of multi-threading approaches in the software design can provide a valuable tool for performance optimization, especially when the different components run asynchronously.

To ensure data security, each software process can establish dedicated UDP communication ports with the other software components and these connections can be maintained open throughout the code execution. The local ports can be closed when the Cortimo Suite is shut down or when the operator chooses to shut down the connections using the GUI.

### VR Arm Application Communication Parameters

The VR arm application can include different modes. 1) The application can be controlled by commands sent by Cortimo; 2) the application can be linked to the wearable device actual position, in this case the application can be controlled by the Bluetooth Controller; and 3) the application can be used as a training/test tool. In this case, the VR arm can be synchronized with other signals and applications via the Cortimo Suite and provide data stream that can be used to train/test the system. In this operation mode, the VR arm can respond to control commands sent from Cortimo Suite with the complete data stream or the application can write to a data buffer the up-to-date protocol information at regular time intervals. In this case, the Cortimo Suite can read the info from the buffer at variable time intervals and store the data stream in a binary file.

### Cortimo Communication Parameters

The Cortimo software can either send/receive data from an external app or read updated information from a UDP buffer. These different data access modes can be kept separate using different UDP ports. The Matlab Cortimo Suite can also communicate with two applications: the Bluetooth Controller System and the VR arm application.

### Wearable Device Command Layer

In order to establish an efficient communication channel between the Cortimo Suite and the wearable device (e.g., the MyoPro device) while ensuring patient safety and data security, a dedicated command protocol layer can be implemented. The Bluetooth Controller Software Module can interpret commands received from other Cortimo applications and stream to the wearable device firmware a subset of verified commands. Moreover, the wearable device API commands and protocols can be closed to external applications. Thus, the Bluetooth Controller Software Module can provide a verified and secure interface layer between the wearable device firmware and the Cortimo Suite.

In some cases, the internal Cortimo command layer for wearable device can consist of two ASCII characters that are embedded in a data packet and that is interpretable by the wearable device backend.

In addition, during wirelessly controlled brace operations, the communication layer can send real-time commands to the wearable device motors using a sequence of characters. For example, the first character can determine the desired movement of the hand motor, while the second character can determine the desired movement of the elbow motor.

### Brace Motor Controller

The Bluetooth Controller Software Module can include dedicated classes that convert Cortimo commands into simple instructions to be sent to the wearable device firmware. Furthermore, the control approach, whether discrete or continuous, can determine how these interface classes operate. Specifically, in the case of discrete control, the Control_Logic class can read the motion request from the BCI decoder and based on its value, act on the current motor position (which the interface can receive in real-time) by applying a discrete increment or decrement. In addition, the Control_Logic class can disregard motion requests that fall outside the range of motion of the device, for instance a request of further flexion, when the brace is fully flexed already can be ignored.

In the case of continuous trajectory control, the Control_Logic class can receive the BCI decoder output as the desired position in angles. This position can be converted into the motor total range of motion percentage before being sent to the wearable device firmware and the actuators.

### Cortimo Output Motor Commands

The Cortimo Suite can output a verified sequence of commands that are sent to the wearable device. Based on the chosen BCI motor control strategy, these commands might either a discrete command or a continuous command representing the precise joint angle rotation expressed in total range of motion percentage. The commands can be stored as two doubles in the Cortimo Application and sent to the Bluetooth Controller Software Module and the VR App as two consecutive ASCII characters. Once received, the two ASCII characters can be converted into required positions and stored into an array of two integer variables.

### File Data Structure

The Cortimo Suite can generate several types of output files. These files can store information relative to the different data streams and system components. They are an integral component of the offline analysis for system optimization and machine learning algorithm training. The output files can be grouped in three categories: NSP brain signal raw data files, Cortimo Binary files, and Cortimo Settings Files.

NSP files can contain all the data and information derived from the neurophysiological acquisition systems (e.g., Blackrock NSP acquisition system, electrocardiogram system, galvanic skin response system, and the like). They can be raw data files and be manipulated and utilized to re-run, simulate or offline analyze the BCI data. These files can store two types of time series: the continuous local Field Potentials (LFPs) and the time stamps of the neuronal spikes detected in real-time by the NSP system.

Cortimo Binary files can store information that the Cortimo Suite collects during real-time BCI sessions. Specifically, data from all the system components can be unified using the unique time stamps from the NSP and stored in binary files. These binary files can be loaded in the offline analysis tool and provide critical data for training the decoders, evaluate performance and identify optimal BCI parameters.

Cortimo Setting files can store the information and settings for the Cortimo decoders and the Cortimo Application. Settings files can be generated in both offline and real-time analysis and contain the information and parameters for the BCI. These files can be re-loaded multiple times and can provide critical information regarding the different BCI sessions and classification performance.

### Binary File Data Structure

The Cortimo Suite can generate proprietary binary data files that are important to collect all the data and information that are generated in a BCI session. The system can generate different types files, depending on the user-selected option and settings of the BCI application.

### Training Data Files

The Cortimo BCI can store training data files for each BCI session.

### LFP Feature Files

The Cortimo BCI can store the LFP features that are extracted in a real-time session. Furthermore, the system can store settings and parameters regarding the extracted features for further processing.

### Spike Rate Feature Files

The Cortimo BCI can store the spike rate features that are extracted in a real-time session. Furthermore, the system can store settings and parameters regarding the extracted features for further processing.

### Cortimo Decoder Settings

The Cortimo Suite can save and load the system and classification settings. These parameters can be stored in dedicated files and be loaded using the GUI. The GUI can assist the operator to monitor the BCI analysis parameters, extracted features and classification methods implemented. In addition to providing visual feedback for BCI settings, these files can store information necessary to run BCI sessions. For example, after launching the Cortimo system, one of these files can be loaded to run a complete BCI sessions end-to-end without any additional user input.

### Decoding Strategies

The analysis parameters and strategies can be changed in real-time and be implemented with a few clicks via the dedicated GUIs. This allows for fast and reliable performance optimization and re-training. This is an innovative approach compared to the traditional BCI algorithms where the entire signal processing cascade and classification are hard-coded in the system and require software programming tools to be modified. The Cortimo main components of the decoding strategies are feature extraction, feature optimization and classification optimization/training.

### Feature Extraction

At runtime, the Cortimo system can conduct feature extraction based on the operator's preferences. Specifically, the operator can select whether to derive features from the LFPs or the spike rates or both. Furthermore, the operator can select the specific NSP channels to be used and the specific time analysis windows and frequency parameters that are going to be extracted.

An additional feature extraction can be implemented by combining features from LFPs and spikes. This approach can extract a certain number of features from the LFPs and combine them with a certain number of features extracted from the current spike rate and the average of the past ten spike rate bins. This approach can provide a feature set update at the rate selected for the LFPs, but it can also take into account the faster data provided by the spike counts.

### Feature Optimization

The Cortimo Suite can conduct feature extraction at run time. These features can be directly selected by the operator and used to quickly train new classifiers and run the system.

Alternatively, the Cortimo Suite can run a complete offline optimization routine, that can operate on both the raw data files and the binary files containing features. This offline optimization toolset can ensure that while more and more training data sets become available, the system can be consistently re-trained and thus, optimal features and classifiers can be selected. This optimization procedure loads raw data files and training data files as selected by the operator and generates a series of performance metrics and settings files that can be quickly reviewed and loaded into the Cortimo system for runtime use.

### Classification Optimization

The Cortimo can introduce classification models that are ready to be trained using either the extracted online features for quick training and testing or the raw NSP data files with the Cortimo BCI training labels for slower but more accurate training and testing. Either choice can generate a settings file that is ready to be loaded in the Cortimo runtime GUI. Furthermore, an optimization routine can be run when new data sets become available and thus the appropriate and optimal models can be re-trained without the need for coding or modifying the Cortimo Application structure. Different classifiers can be trained, tested and reloaded easily, thus ensuring that the Cortimo Application can quickly switch to a different decoder and implement it. This level of flexibility allows for quick system adjustment to specific experimental and environmental conditions. For example, seven retrainable classification algorithms that are supported by the Cortimo Suite are: Linear Discriminant Analysis, Coarse Decision Tree, Quadratic Support Vector Machine, Linear Support Vector Machine, Medium KNN, and Ensembled Bagged Tree.

These models can be trained using the data collected by the Cortimo. Specifically, NSP raw data files or binary feature files can efficiently be combined with the training/label binary data files collected by the BCI system. By using unique and very precise time stamps for automatic labeling of the brain derived signals, the system can group the data into specific epochs, subsequently, it can extract features from the epoched signals and associate them with the correct output labels. At the end of this process a large training data set is available for optimization of the above-mentioned classification models. Once these models have been trained, a 3-fold cross-validation method can be applied to evaluate the performance of each trained model and the selected features. Finally, the Cortimo system can generate a report with summary performance metrics. The operator can choose whether to adopt the suggested best-performing decoder or other decoders, based on different considerations. Based on the operator selection, the appropriate settings files containing all the necessary information for implementing the decoder are generated and loaded into the Cortimo GUI. Once a new or an existing settings file is loaded the Cortimo BCI is ready for runtime use.

Further, the Cortimo Suite can implement separate decoders, one for each joint to be controlled (e.g., hand and elbow). These decoders can be different and rely on different extracted features. In other words, the two joint commands can be derived using parallel but independent signal processing and decoding algorithms.

### Component Integration and Safety

All the Cortimo software components are designed to not alter the safety and principle of operation of the existing FDA-cleared software components. Furthermore, the system has been designed to only operate when all the components are properly running, in other words, the malfunction of a single integrated component can cause the software real-time control to prompt a series of warnings and messages for the operators to manage the situation.

### Manuscript 1

Stroke is a leading cause of disability with a global prevalence of over 42 million people in 2015, affecting over four million adults in the United States alone with 800,000 new cases per year. Stroke leads to permanent motor disabilities in 80% of cases, and half of stroke survivors require long term care. Brain computer interface (BCI) technologies offer a potential solution to restore functional independence and improve health in people living with its effects. In the past decade, intracortical BCI technology has continued to advance, with multiple groups demonstrating the safety and efficacy of this approach to derive control signals to restore communication and control. In parallel, wearable robotic orthosis technology can benefit patients with weakened limbs. This single-patient pilot clinical trial sought to prove that a commercially available powered arm orthosis could be linked to the cerebral cortex in an adult with the most common form of chronic stroke. A direct path from the brain's motor centers to the orthotic could reanimate a paralyzed limb to enable useful hand and arm function.

Several signal sources have been coopted to provide commands to move paralyzed limbs. Electromyographic (EMG) control of a powered orthosis or functional electrical stimulation (FES) of muscles, has proven problematic either because users could not generate sufficient or reliable activity to provide a good control signal, or because voluntary activation of those recorded muscles (that were intended to generate the command) was opposed by the stimulator's effects. Contralaterally controlled electrical stimulation—where activity from the unaffected arm triggers stimulation on the paretic arm—is a useful therapeutic intervention to improve function in the weaker limb but it is not clear how this unnatural command source could be generalized to continuously-worn devices that enable independent arm movements. Several groups have explored scalp EEG, which is closer to the command's origins, to derive control signals to drive robotic braces, and in one case, FES. While using EEG-derived signals may be promising for rehabilitation therapy, it would not be feasible for daily independent function because skin sweat and hair can cause impedances to fluctuate, compromising signal quality. Daily application of even a subset of contacts to the same skin sites can lead to skin breakdown and cellulitis. Further, EEG signals are limited in the commands that can be easily and reliably derived from the available signal. By contrast, intracortical interfaces offer a rich sources of high resolution, multidimensional control signals, since it is the origin of such signals in healthy adults, in non-human primates and in people with spinal or brainstem disorders.

While the vast majority of strokes involve cerebral white matter and even direct parenchymal damage, intracortical neuromotor prosthetics have not been tested in people with strokes above the mesencephalon. It is not known whether motor cortex remains a reliable signal source in this large population. A proof-of-concept that a brain-computer interface, based on micro-electrode arrays implanted in intact cortex above a subcortical stroke, could restore behaviorally useful independent, voluntary movement, could lead to the development of a fully implantable medical device that, in principle, could reverse the motor deficits caused by stroke.

### Methods

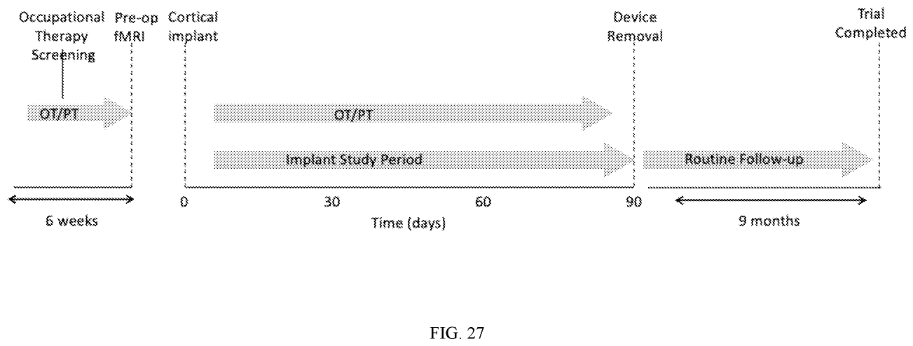

Approval for this study was granted by the US Food and Drug Administration (Investigational Device Exemption) and the Thomas Jefferson University Institutional Review Board. The participant described in this report has provided permission for photographs, videos and portions of his protected health information to be published for scientific and educational purposes. After completion of informed consent, medical and surgical screening procedures, two MultiPorts (Blackrock Microsystems, UT), each comprising two 8×8 platinum tipped microelectrode arrays tethered to a titanium pedestal connector, were implanted into the cortex of the precentral gyms using a pneumatic insertion technique. Details of the human surgical procedure are in preparation for publication and followed other similar studies. Trial selection criteria are available online (see Clinicaltrials.gov, NCT03913286). The trial was designed with the implantation phase to last a maximum of three months (FIG. 27).

### Participant

The participant was a right-handed male who experienced right hemispheric stroke, manifest as acute onset dense left hemiparesis and expressive aphasia, at which time he was age between ages 35 to 40. Due to unknown time of onset and hypertension at presentation, the participant was not a candidate for thrombolysis. CT angiogram showed occlusion of the right posterior cerebral artery and high-grade stenosis of the left posterior cerebral artery in the proximal P2 segment. MRI of the brain showed acute infarcts in the right basal ganglia/corona radiata and right occipital lobe. He was started on dual antiplatelet therapy for 3 weeks and then was transitioned to aspirin 81 mg once daily, along with atorvastatin and anti-hypertensives. He had left-sided hemiparesis, dysphagia, left homonymous hemianopsia and dense left visual neglect and was transferred for inpatient rehabilitation. Over a period of three months, aphasia and dysphagia resolved and he learned to ambulate independently, albeit with a persistent left foot drop. Neuroimaging showed evidence of multi-focal strokes, and prior silent strokes. The participant had previously been in good health and did not have any known stroke risk factors such as diabetes or smoking. There was a history of loud snoring and the participant had not been evaluated for obstructive sleep apnea. Transthoracic and transesophageal echocardiography were normal as were serial hypercoagulability panels; the participant was adopted and the biological family history unknown. The participant was deemed to have had embolic strokes of unknown source. Although serial electrocardiography since the stroke was normal, the participant is being scheduled for a loop recorder to survey for possible paroxysmal atrial fibrillation. The participant had learning disabilities and was presumed to have had mild cognitive impairment prior to the stroke. Screening formal neuropsychological testing identified neurocognitive problems (full scale IQ 59) and also concluded that the participant remained fully capable to provide proper informed consent and to participate in this trial, meeting its demands and requirements. The participant provided both verbal and written informed consent, both to participate in the trial and to share his identifying information with the public. He had been working full time at the time of the stroke and has been unable to return to work since the stroke.

Pre-Operative fMRI

The participant underwent MM on a 3T Philips Ingenia MM scanner. A 1 mm isotropic 3-D T2 FLAIR was obtained for structural localization. A single-shot echoplanar gradient echo imaging sequence with 80 volumes, repetition time (TR)=2 s, echo time (TE)=25 msec, voxel size=3×3 mm2, slice thickness=3 mm, axial slices=37. The participant was asked to visualize movements of his paretic left hand during the MM. Each motor trial consisted of a block design featuring a 20 s rest block and a 20 s active block repeated. This block design was repeated between 4 times for a total of 240 s scans. Visual stimuli comprised a 20 s video depicting a 3D modeled limb at rest, followed by a 20 s video of the limb performing the desired task. Motor tasks included repeated hand open/clench or arm extension elbow, and were either “active” (participant performed or attempted to perform motion) or “passive” (physician manually moved participant's arm). In active tasks, the participant was instructed to follow the movements in the video or concentrate on following for the paretic limb. Task prioritization was based on pre-exam training of the participant's capabilities and examination of BOLD activation observed during the scan. Post processing including motion correction, smoothing, and general linear model estimation performed using SPM software (www.fil.ion.ucl.ac.uk/spm) and Nordic brain EX software (NordicNeuroLab, Bergen, Norway). Statistical maps were overlaid on the 3D T2 FLAIR image for visualization of activation.

### Cortimo System

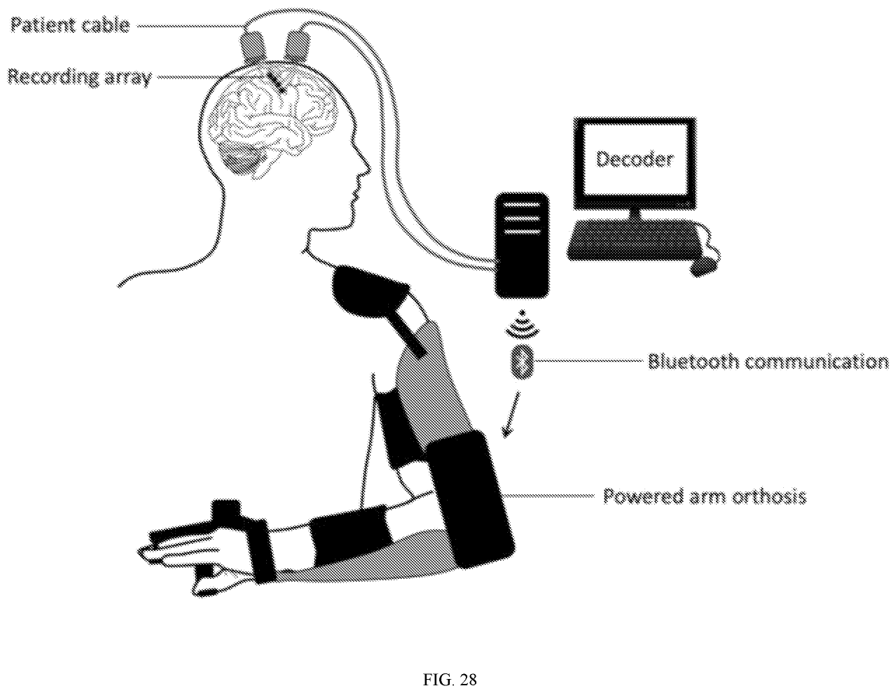

‘Cortimo’ is the designation provided to the FDA to represent the overall system (FIG. 28) that comprised two percutaneous Multiports (Blackrock Microsystems), each in turn having two multi-electrode array sensors, the cabling, amplifiers, software and the powered MyoPro orthosis. Each sensor is an 8×8 array of silicon microelectrodes that protrude 1.5 mm from a 3.3×3.3-mm platform. At manufacture, electrodes had an impedance ranging between 70 KOhm and 340 KOhm. The arrays were implanted onto the surface of the MI arm/hand region guided by the pre-operative fMRI; with electrodes penetrating into the cortex to attempt to record neurons in layer V. Recorded electrical signals pass externally through a Ti percutaneous connector secured to the skull. Cabling attached to the connector during recording sessions routes signals to external amplifiers and a computer that process the signals and convert them into different outputs, such as servo motor position of the MyoPro brace or screen position of a neural cursor. Currently, this system must be set up and managed by an experienced technician.

### MyoPro Brace

The MyoPro (Myomo, Inc, Cambridge, Mass.) is an FDA-cleared myoelectric powered arm orthosis designed to support a paretic arm. The rigid brace incorporates metal contacts attached to soft straps that can be adjusted such that contacts rest on the biceps and triceps proximally, and on wrist flexors and extensors distally, on the paretic upper extremity. The sensors continuously record the root mean square of underlying muscle activity. Thresholds are manually set such that signals exceeding them will trigger one of the MyoPro motors. Because the participant retained residual elbow flexion and extension strength, the motor at the elbow was set up such that biceps activation triggered elbow flexion and triceps activation triggered elbow extension. For hand opening, the MyoPro was set up to either use myoelectric control, or to use BCI-based control. Since the participant was unable to voluntarily extend the wrist or open the fingers, the myoelectric mode was set up such that the default state was with the hand open, and it would only be closed by activating enough wrist flexor activity.

### Recording Sessions

Research sessions were scheduled five days per week at a temporary residence, adjacent to the hospital, provided to the participant. Sessions could be cancelled or ended early at the participant's request. Sessions would commence with neural recording and spike discrimination. While initial sessions included filter building and structured clinical end-point (cursor control) trials, in the final month of the trial, “training-less” algorithms were used with the participant proceeding directly to BCI-controlled hand action once patient cables were connected. Performance of computer tasks, orthosis control and occupational therapy exercises followed. The electrodes and neural signals selected immediately before filter building remained constant for any given session's orthosis control trials.

### Decoder Filter Building

Units were extracted using an automatic thresholding approach based for each electrode channel, based on Root Mean Squared multipliers. For each session, single and multiunit data or high frequency (100-1000 Hz) local field potentials derived from multiple channels (20-30) were used to create a linear filter to convert these real-time multidimensional neural features into either a one or a two-dimensional (position or velocity) output signal. Motor activity and motor imagery approaches were tested for filter building, including imagining opening and closing the paretic hand, passively flexing and extending the elbow, passively opening and closing the hand, and observing a computer cursor displayed on a monitor moving up and down without any specific instruction. Training data for building the linear filter were collected with the participant gazing at a screen where a target cursor was moved slowly up and down for one minute (5 seconds to go from the top to the bottom of the screen or vice versa, at 20° visual angle). After this preliminary filter was built, a new 1-minute re-training session was performed, this time the manually controlled target cursor was accompanied by a prediction cursor that was neurally controlled by the participant. Using this additional training set, a second filter was built and then tested on a simple target acquisition game in which the y-position of the predicted output was discretized into zones such that positions on the upper part of the screen would cause an animation sprite to move up by a fixed distance (1 cm), and positions on the lower part of the screen would cause the sprite to move down by the same fixed distance.

### BCI Orthosis Use

The discrete output was then used to control the aperture of the hand via the MyoPro's hand brace motor. The up-down mapping on the screen was translated into closed-open positions of the hand. The participant then performed a series of functional tasks including grasping and then dropping an object, the Action Research Arm Test, and a variation on Jebsen Taylor item moving test. These were tested with both the participant seated and standing.

### “Training-Less” Mapping

When the participant would attempt to overpower the orthosis motors with residual finger flexion strength, a novel ‘training-less’ approach was deployed in which a rolling 1-second baseline of the LFPs signals was used to calculate spectral power in the high gamma band (100-500 Hz). Namely, 1-second long LFP continuous voltages were used for computing the average spectral density estimation in the frequency band 100-500 Hz, using non-overlapping frequency bins with a 50 Hz width. Spectral density was computed using the Matlab periodogram method. Values were updated every 500 ms, using 1-second-long rolling windows with 50% overlap. Real-time spectral features derived from the 20 most neuromodulated channels were averaged across channels to produce a single high gamma band value for each 500 ms software update. Orthosis hand-closure would be triggered by an increase in this mean spectral power from the resting baseline ranging between 0.5 and 3 V²/Hz to values greater than 10 V²/Hz, where real-time values above this threshold would make the hand motor close.

### Concomitant Occupational and Physical Therapy

Since being discharged from acute rehabilitation 60 days after the initial stroke, the participant enrolled in outpatient physical and occupational therapy. Prior to the device implantation, the participant completed a six-week course of occupational therapy screening phase. Following device implantation, the participant continued occupational therapy, twice per week, and physical therapy, once per week. Occupational therapy focused on postural training while seated and walking, donning and doffing the MyoPro, and using the MyoPro for functional activities. Timed functional electrical stimulation (e.g., pincer grasp programs; XCite, Restorative Therapies) and vibration therapy were used for spasticity management. Physical therapy exercises included scapular mobilization, progressive range of motion, weight bearing, forced use with game-related activities to encourage left UE volitional control, and aerobic endurance exercise.

### Results

The participant underwent intracortical implantation in autumn of 2020 and explanation three months later on January 2021, in accordance with the intended 3-month duration of the trial. Over the course of the study, the participant had three minor, and one serious, device-related adverse events, all of which were treated, resolved, and reported to the governing regulatory bodies. The serious adverse event was the development of a scalp infection at the left pedestal site one week prior to the device removal date, despite a regimen of topical antibiotics and regular cleaning. This infection was anticipated and was described as a potential risk in the informed consent form and consent interview. The left pedestal site had posed a challenge since the time of the initial surgery as it was not possible to exactly re-approximate the skin flap leaving the base of the pedestal exposed. This area was protected and granulated and grew new skin. The participant was afebrile and asymptomatic, and the infection was detected only by close visual inspection. The participant was treated with twice daily antibiotic for the 7 days prior to the device removal. Pedestal site skin cultures taken at device removal revealed pansensitive staphylococcus lugdunesis and staphylococcus capitis, and yeast, and appropriate antimicrobial treatment was provided. No organisms grew from cultures taken of adjacent bone. The only macroscopic evidence of infection at device removal was a small area (˜2 cm³) of erythema and friable tissue at the skin adjacent to the right pedestal. The participant was discharged home. The participant remains in the Cortimo trial for ongoing neurosurgical follow-up and surveillance, and to track any further performance improvements in myoelectric MyoPro use with ongoing outpatient occupational therapy.

### Preoperative Anatomic and Functional Neuroimaging

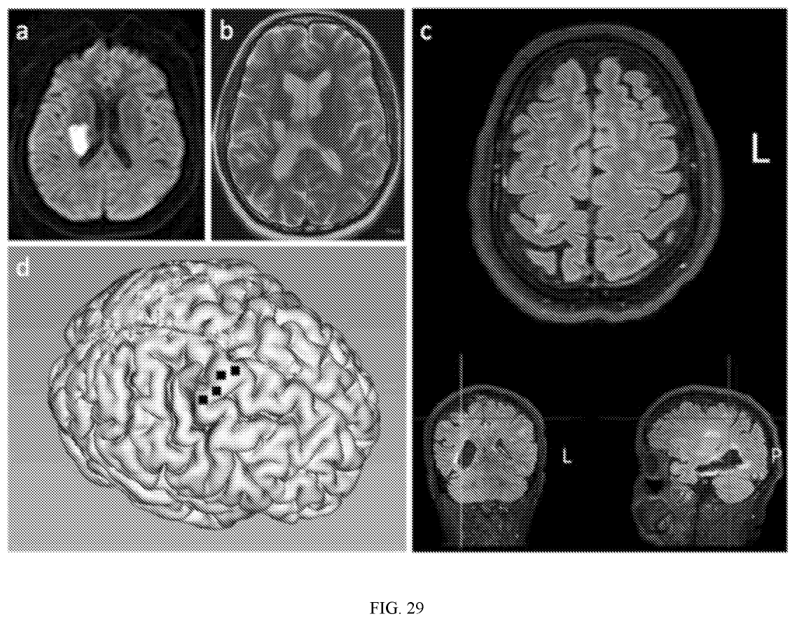

Preoperative imaging revealed the old infarct in right lentiform nucleus and adjacent white matter including corona radiata and a portion of the posterior limb of the internal capsule, along with a large old right PCA infarct, progressed since the acute stroke imaging MRI from 2019 (FIG. 29). In addition, a small region of bandlike signal abnormality involving subcortical white matter and medial aspect of hand knob region of right precentral gyms was identified, likely reflecting retrograde neuronal degeneration. On DTI, there was extensive loss of fractional anisotropy in the region of right corticospinal tract from old infarct. The “imagined” left hand motor paradigm and passive motor paradigm were diagnostic with good concordance. Subsequent to hypercapnia challenge, a BOLD signal was evident at the precentral gyms. On the “imagined” left hand motor paradigm, activation was noted in the expected location along central sulcus involving lateral aspect of the hand knob region of the precentral gyms and the adjacent portion of postcentral gyms (FIG. 29C). On the passive left elbow motor paradigm, activation was seen along central sulcus which shows good concordance with the “imagined” motor task as discussed with a slightly more posterior and superior extension of activation reflecting the prominent sensory component of this passive motor paradigm. A 3D brain model was printed using the 3D FLAIR sequence to allow for 3D visualization of the surgical field for more accurate pre-operative planning (FIG. 29D).

### Neural Recordings

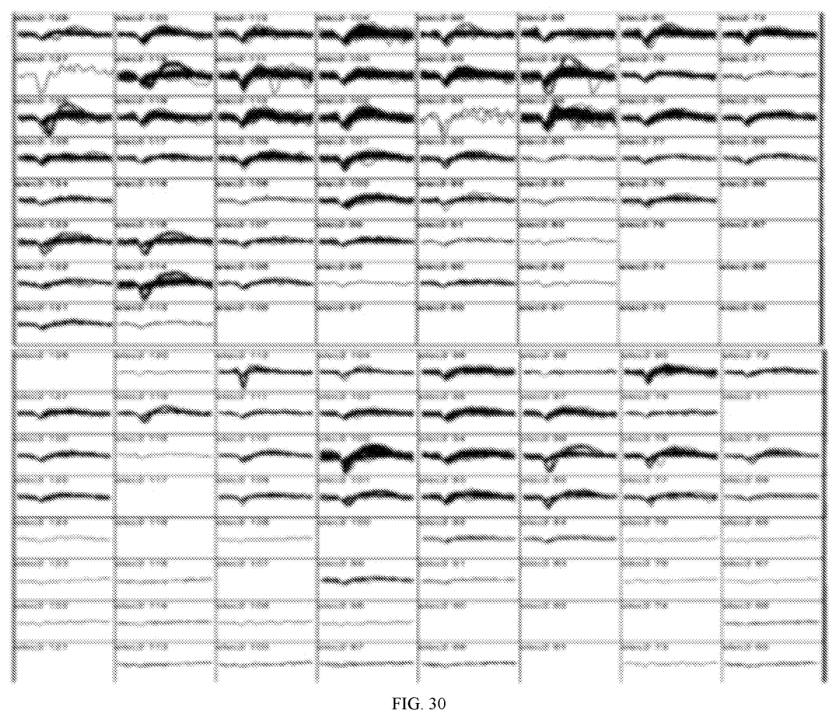

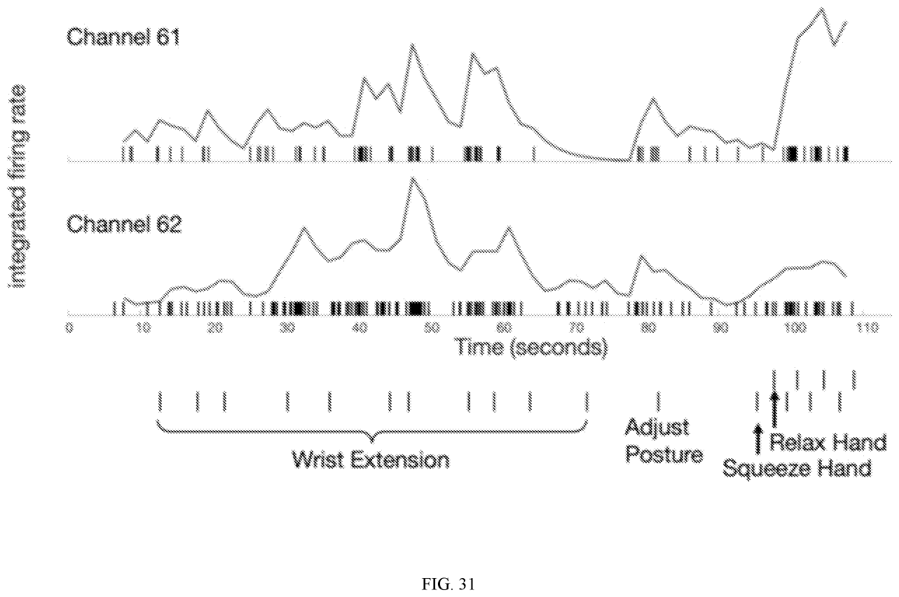

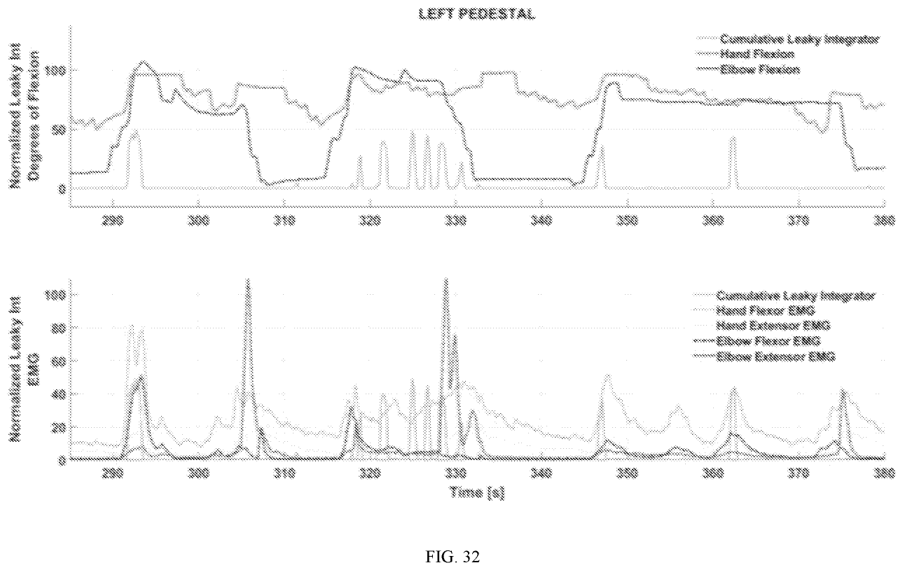

Well-delineated single units were recorded from 87 of the 256 channels (FIG. 30). Neural activity correlated with actual and attempted movements in both the paretic left arm in addition to the intact right arm. The discharge rate of various units appeared to correlate with specific residual actions, including the wrist extension that gradually developed in the course of the three-month duration (FIG. 31). By taking the spike counts recorded at each channel every 200 milliseconds and running them through a leaky integrator, and then summing these leaky integrator outputs across all channels, we were able to visualize the cumulative cross-array firing rate activity in comparison to forearm electromyographic activity (FIG. 31). Of the 256 electrodes, in each session, we identified 40 channels that were eventually used for neural decoding. These channels were used for extraction of neural features that coded for hand and elbow flexion and extension. Two main hand open-close decoding approaches were used: 1) A discrete two-state classifier based on a 1-dimensional linear filter continuous output; 2) a “training-less” threshold crossing approach with a rolling baseline normalization.

### Orthosis Control

The left upper extremity score on the Action Research Arm Test (ARAT) was 0 without the orthosis on, 5 using the orthosis under myoelectric control, and 10 using the orthosis under direct brain-control. In one component of the Jebsen-Taylor standardized test of hand function, the goal is to pick up and move 5 cans, one at a time, a few inches away forward on the table (normal times are 3.23 seconds for empty soup cans in subtest 6, and 3.30 seconds for full cans in subtest 7). Because the design of the hand orthosis precluded the ability to grasp a soup can (e.g., the brace only supports the thumb and next two digits), the participant performed variations on the test. It took the participant 146 seconds to pick up, move and release 5 pill bottles using myoelectric control, and 95 seconds to perform the identical task under BCI control. Another task was to hold an object in the right hand (e.g., a stress ball or a whiteboard eraser) and place it into the paretic left hand, and then extend the left arm down towards the floor and drop the object into a bin; this process was then repeated 5 times in a row. Both these tasks were performed with the participant seated. On two trials of this pick-up-and-drop-5 in myoelectric mode, the participant's completion times were 128 and 222 sec/trial; seconds; on five trials of the same task in BCI mode, times were 81, 106, 137, 214 seconds. In addition to measuring the total time to perform these grasp-move-release tasks, we also quantified the time it took to release an object once the hand was in the target position. Hand release times were faster under BCI control than myoelectric control (p=0.04, two-sample t-test).

### Motor Outcomes

Motor measures, performed when the participant was not connected to the BCI or wearing the MyoPro orthosis, were tracked over time and demonstrated that the implantation procedure did not decrease residual strength in the paretic left arm and left leg. In fact, muscle strength increased in the left arm. Whereas serial neurological exams since the time of the stroke demonstrated an absence of voluntary wrist extension or finger extension (0/5 on manual muscle testing, starting two months into the trial, the participant began to consistently exhibit voluntary wrist extension against gravity (3/5), and on a few occasions was able to voluntarily extend the fingers slightly (2/5). One month prior to the device implantation, the Fugl-Meyer upper extremity score was 30 (out of a maximum of 66) for the left upper extremity; this increased to a score of 36 four weeks after the two Multiports were implanted, and a score of 38 seven weeks post-implantation. Although the participant did not receive botulinum toxin injections, or receive any type of anti-spasticity medication, during the clinical trial, spasticity gradually decreased with time as reflected in gradually decreasing numbers on serial measurements of the modified Ashworth scale for spasticity for passive flexion and extension movements of the fingers, wrist, and elbow, along with internal and external rotation of the shoulder.

### Discussion

This pilot trial demonstrated that ensemble single unit activity remains active in ipsilesional cerebral cortex overlying chronic subcortical stroke. To our knowledge, this is the first report of intracortical recordings in ipsilesional cerebral cortex for a stroke above the mesencephalon. The trial established that single neuron, movement related activity can be decoded used to control a powered orthosis that restored functionally useful voluntary upper extremity movement. Importantly, this brain-computer interface system can be used simultaneously with residual intact movement, in particular in a limb with a gradient of intact to absent voluntary movement, as is common following cerebral strokes. While myoelectric approaches based upon wrist flexion did enable voluntary hand opening, this approach triggered increased muscle tone that subsequently slowed orthosis use (as the motors were opposing the abnormal tone): the BCI control mode essentially bypassed this issue and allowed motors to operate more smoothly and quickly. Electromyographic recordings demonstrated that while the participant did continue to engage wrist flexors during BCI control, the amplitude was decreased from abnormally elevated levels to more normal amplitudes.

This trial was not intended to restore voluntary motor control in the hemiparetic upper extremity in the absence of any device use, but even so, we found that strength improved, and spasticity decreased. This suggests that the implantation of four arrays into ipsilesional cortex did not exacerbate pre-existing hemiparesis (i.e., it did not worsen hand or arm weakness); indeed, after the intervention hand functions improved. One potential explanation for the unexpected improvements in voluntary wrist and finger extension is mass practice. Another, more speculative, explanation for the participant's improved forearm function is that the daily exercise of ipsilesional cortical activity for BCI-orthosis control, promoted a plasticity driven response to either normalize or compensate for abnormal motor synergies.

Although the limited number of trials on various tasks reduced statistical power to compare myoelectric to BCI control, qualitatively there appeared to be a trend of faster control in the BCI mode. This may be due to the fact that triggering orthosis action from direct cortical recordings does not activate abnormal forearm synergies in the same manner that myoelectric control appears to. Spasticity may represent abnormal plasticity and loss of corticoreticular facilitation of the medullary inhibition center leading to decreased inhibition from the dorsal reticulospinal tract on the spinal stretch reflex: the medial reticulospinal and vestibulospinal tracts are unopposed leading to stretch reflex hyperexcitability. In the myoelectric mode, where hand closing is triggered by activation of residual wrist flexors, this hyperexcitability is inevitably triggered such that the orthosis motors have to ‘fight harder’ to open the hand, slowing that process. In the BCI mode, even if residual wrist flexor and extensor activity are engaged, it is to lesser degree such that abnormal tone is not elevated, and the orthosis motors can more easily and rapidly achieve hand actions.

This pilot study implies that usable control signals are present in ipsilesional cerebral cortical activity. To be clinically scalable, future devices must be fully implantable to minimize infection risk and allow mobility. With the advent of fully implantable BCI (i.e., no percutaneous connectors), a wider range of stroke survivors could benefit. An option that may gain even wider clinical adoption would be to couple direct cortical control to implantable functional electrical stimulation in the paretic arm, as has been demonstrated in at least one person with chronic stroke. Direct cortically driven peripheral muscular stimulation may have both rehabilitative and direct functional benefits if deployed continuously in daily life. Fully implantable brain-computer interfaces may represent a medical device opportunity to help stroke patients break through their plateau in recovery and to achieve greater functional independence.

### EQUIVALENTS

Although preferred embodiments of the invention have been described using specific terms, such description is for illustrative purposes only, and it is to be understood that changes and variations may be made without departing from the spirit or scope of the following claims. The inventors further require that the scope accorded their claims be in accordance with the broadest possible construction available under the law as it exists on the date of filing hereof (and of any application from which this application obtains priority,) and that no narrowing of the scope of the appended claims be permitted due to changes in the law (either statutory or judge-made) subsequent to the priority date hereof.

### INCORPORATION BY REFERENCE

The entire contents of all patents, published patent applications, and other references cited herein are hereby expressly incorporated herein in their entireties by reference.

1. A computer-implemented method for assisted actuation of a patient movement, comprising:
receiving a set of neural signals from a set of neural sensors;
extracting a set of features from the set of neural signals;
inputting the set of features into a classification model;
determining from the classification model an attempted activity of a user; and
transmitting a set of stimulation signals to one or more output effectors according to the attempted activity and the set of neural signals.

2. The computer-implemented method of claim 1, further comprising:
training the classification model, wherein the training comprises:
receiving a set of training neural signals from the set of neural sensors;
receiving input indicative of an action performed by a trainer;
extracting a set of training features from the set of training neural signals; and
mapping the set of training features to the indicative action.

3. The computer-implemented method of claim 2, wherein training the classification model further comprises determining a feature value threshold from the mapping, wherein the attempted activity of the user is further determined from a feature of the set of features reaching the feature value threshold.

4. The computer-implemented method of claim 2, wherein the trainer comprises the user, a provider of physical therapy, a provider of occupational therapy, or a combination thereof.

5. The computer-implemented method of claim 1, further comprising:
identifying a set of proportional values between the set of neural signals and the attempted activity of the user; and
generating the set of stimulation signals according to the set of proportional values.

6. The computer-implemented method of claim 5, wherein the identifying the set of proportional values is effected via a multilayer perceptron network, a convolution neural network, a genetic algorithm, a binary particle swarm optimization process, a generative adversarial network, a support vector machine of polynomial and radial basis kernel, a Kalman filter, a generalized linear mixed model, a particle filters, a random forest algorithm, a rotation forest algorithm, or a combination thereof.

7. The computer-implemented method of claim 1, further comprising:
identifying an activation pattern from the received neural signals, wherein determining the attempted activity is according to the identified activation pattern.

8. The computer-implemented method of claim 1, further comprising:
modifying the classification model according to the set of features.

9. The computer-implemented method of claim 1, wherein the set of neural signals comprises scalp EEG, subgaleal EEG, intraosseous EEG, epidural EEG, subdural EEG, intracortical LFPs, depth EEG, single unit recordings, heart rate, heart rate variability, respiratory rate, galvanic skin conductance, blood sugar level, pupil diameter, extraoculogram, electromyogram, positioning of a user body part, user's kinematic and kinetic signals, sound signals, keyboard entry, mouse click, joystick use, or a combination thereof.

10. The computer-implemented method of claim 1, wherein the output effector comprises a set of electrical contacts, an electrical prosthetic, a brain-computer interface, or a combination thereof.

11. A system comprising:
a neural signal processor configured to:
receive a set of brain signals from a user;
digitize the set of brain signals; and
store the digitized brain signals in a buffer;
a neural signal analyzer configured to:
retrieve the digitized brain signals from the buffer;
identify a set of spike counts, local field potentials (LFPs), or a combination thereof, from the digitized brain signals;
extract a set of features from the set of spike counts and LFPs;
input the set of features into a classification model;
identify from the classification model an attempted motor movement of the user;
generate a motor control command according to the attempted motor movement; and
transmit the motor control command; and
a rehabilitation prosthetic configured to:
receive the motor control command; and
generate a corresponding motor movement according to the motor control command.
